(19) United States
(12) Patent Application Publication
(10) Pub. No.: US 2017/0247690 A1
(43) Pub. Date: Aug. 31, 2017

(54) ONCOVIRAL TREATMENT WITH NUCLEASE AND CHEMOTHERAPEUTIC

(71) Applicant: Agenovir Corporation, South San Francisco, CA (US)

(72) Inventors: Stephen R. Quake, Stanford, CA (US); Derek D. Sloan, Belmont, CA (US)

(21) Appl. No.: 15/442,020

(22) Filed: Feb. 24, 2017

Related U.S. Application Data
(60) Provisional application No. 62/299,792, filed on Feb. 25, 2016.

(51) Int. Cl. C12N 15/11; A61K 38/05; A61K 45/06; C07K 16/28; C07K 16/22; A61K 38/20; A61K 38/46; A61K 39/395

(52) U.S. Cl. CPC C12N 15/11; C12Y 301/00; A61K 38/465; A61K 38/05; A61K 39/3955; C07K 16/2887; C07K 16/22; C07K 16/2803; A61K 38/2013; A61K 38/2073; A61K 38/2086; A61K 45/06; C12N 2310/20; C12N 2320/31; C07K 2317/24; A61K 2039/505

(57) ABSTRACT

Compositions and methods for treating infection-associated cancer include the use of a nuclease that cuts nucleic acid of an oncovirus in combination with an adjunct chemotherapeutic that treats cancerous cells. For example, a Cas9 endonuclease and a guide RNA that matches a target in a viral genome without having any corresponding match in the human genome can be delivered along with an anti-apoptotic inhibitor.

### CROSS-REFERENCE TO RELATED APPLICATION

This application claims benefit of U.S. Provisional Application Ser. No. 62/299,792, filed Feb. 25, 2016, incorporated by reference.

### TECHNICAL FIELD

The invention relates to oncoviruses.

### BACKGROUND

Millions of people die each year from cancer. Evidence shows a link between cancer and infectious disease. In fact, it is understood that infectious disease represents the third leading cause of cancer worldwide. De Flora, 2015, J Prey Med Hyg 56:E15-E20. Unfortunately, viruses and cancer are difficult to successfully treat. Some cancer drugs, for example, may slow the growth of a tumor yet leave behind affected cells that may proliferate again after treatment. Some viruses, including known oncoviruses, exhibit an asymptomatic latent phase during which they present no activity or proteins to target with a treatment.

As such, oncoviruses and their resultant tumors may be some of the most difficult to treat. Even if a cancer drug successfully removes a tumor, latent viral genes may later be expressed if the virus re-enters an active stage of infection. When the virus re-enters the active stage of infection, it may trigger cell proliferation resulting in new tumors such as Burkitt's lymphoma in the case of Epstein-Barr or a squamous cell carcinoma in the case of Human Papilloma Virus.

Even if a viral treatment had good prospects for clearing the infection, cancerous cells may still proliferate. That is, even in cases where an oncoviral infection may have been a causal factor, tumors may continue to grow once the infection is removed.

### SUMMARY

Compositions and methods for treating tumors include a cancer therapeutic (i.e., a drug intended to stop or slow tumor growth or induce cell death in cancer cells) and a nuclease to degrade genetic material in the tumor. The nuclease digests nucleic acid from the tumor genome or from an oncovirus. The nuclease complements the therapeutic effect of the cancer drug. For example, a cancer chemotherapy typically acts to selectively kill tumor cells. However, no therapeutic is 100% effective. In combination with the nuclease, the therapy kills or disables a greater number of cancer cells. Thus, the nuclease adds a layer of protection by preventing proliferation in tumor cells not killed by the cancer drug. Additionally, a nuclease with a mechanism of action orthogonal to that of a cancer therapeutic may have additive or synergistic effects. Thus, combination of an endonuclease with cancer therapeutics may facilitate administering lower dosage of cancer therapeutics, which often have dose-limiting toxicities associated in healthy tissues that have higher division rates, e.g. gut epithelium. Using a nuclease with a cancer drug may be particularly beneficial where a tumor is associated with an infection by an oncovirus, as the cancer drug can cause cell death while the nuclease can cleave viral nuclease preventing recurrence of an active oncoviral infection. Thus it may be preferable to use a nuclease that preferentially cuts oncoviral nucleic acid over human genetic material.

Nucleases that are directed to specific targets include transcription-activator-like effector nucleases (TALENs), meganucleases, zinc-finger nucleases (ZFNs), and CRISPR-associated (Cas) nucleases. Preferred embodiments use a nuclease that may be targeted to oncoviral DNA along with a cancer drug. For example, a Cas9 endonuclease and a guide RNA that matches a target in a viral genome without having any corresponding match in the human genome can be delivered along with an anti-apoptotic inhibitor. For treating an oncovirus such as Epstein-Barr virus (EBV), the guide RNA can program Cas9 to degrade key EBV genes while the chemotherapeutic stops the proliferation of a lymphoma. In another example, human papillomavirus (HPV) can be treated using a targetable nuclease to target genes of the HPV genome and a chemotherapeutic such as cisplatin to trigger cell death in a cervical carcinoma. In another example, merkel cell carcinoma (MCC) associated with merkel cell polyomavirus (MCV) may be targeted with MCV-specific guide RNA in combination with carboplatin and etoposide. Compositions and methods of the invention attack cancers of infectious origin at two defining points: both the causative infecting virus and the cancerous proliferation of cells.

The nuclease may be delivered as an active protein—or ribonucleoprotein in the case of a Cas-type nuclease—or encoded in a vector, such as a plasmid or mRNA, in a viral vector, such as adeno-associated virus (AAV), or in a lipid or solid nanoparticle. The nuclease may be delivered via a pharmaceutically acceptable composition that also includes the cancer drug, or the two may be separately delivered to treat the tumor.

In certain aspects, the invention provides a composition for treating a tumor. The composition includes a cancer drug and a nuclease. The cancer drug may be actinomycin, all-trans retinoic acid, anthracycline, bleomycin, bortezomib, carboplatin, carfilzomib, capecitabine, cisplatin, chlorambucil, cyclophosphamide, cytarabine, daunorubicin, disulfiram, docetaxel, doxifluridine, doxorubicin, epirubicin, epothilone, epoxomicin, etoposide, fluorouracil, gemcitabine, hydroxyurea, idarubicin, imatinib, interferon alpha, irinotecan, ixazomib, lactacystin, mechlorethamine, mercaptopurine, methotrexate, mitoxantrone, oxaliplatin, paclitaxel, pemetrexed, salinosporamide A, tenipo side, topotecan, valrubicin, vinblastine, vincristine, vindesine, vinorelbine, venetoclax. The cancer drug may be a biologic. The cancer drug may be a monoclonal antibody (mAb) that targets cell-specific surface antigens. A suitable monoclonal antibody may include, e.g., rituximab, bevacizumab, or pembrolizumab. Rituximab (Rituxan) may function as an anti-CD20 to deplete B cells. The cancer drug may be an immune checkpoint inhibitors such as, e.g., anti-PD-1 or anti-VEGF. The cancer drug may be a recombinant cytokine such as, for example, Interleukin 2 (IL-2), Interleukin 11 (IL-11), or Interleukin 15 (IL-15). The nuclease may be, for example, an endonuclease, an exonuclease, DNase I, a CRISPR-associated nuclease, Cfp1, a transcription-activator-like effector nuclease, a meganuclease, and a zinc-finger nuclease.

In certain embodiments, the nuclease preferentially cuts nucleic acid of a an oncovirus such as a human papilloma virus (HPV), an Epstein-Barr virus (EBV), Kaposi's sarcoma-associated herpesvirus (KSHV), hepatitis B virus (HBV), hepatitis C virus (HCV), human T-cell lymphotrophic virus type I (HTLV-I), and Merkel cell polyomavirus (MCV). The nuclease may be a CRISPR-associated nuclease, and the composition further include a guide RNA complementary to a portion of the nucleic acid.

The cancer drug may be a proteasome inhibitor such as lactacystin, bortezomib, disulfiram, salinosporamide A, carfilzomib, epoxomicin, and ixazomib. In one embodiment, the nuclease is Cas9 and the oncovirus is Epstein-Barr virus. The cancer drug may be bortezomib.

The composition may include an antiviral treatment such as ganciclovir or Gardasil. The composition may include an epigenetic modifier such as a DNA methyltransferase (DNMT) inhibitor or a histone deacetylase inhibitor (HDI or HDACi), such as vorinostat or panobinostat.

Aspects of the invention provide a composition that includes a cancer drug and a vector nucleic acid, such as a plasmid, encoding a nuclease, wherein the cancer drug and the nuclease are as described above.

Aspects of the invention provide a method for treating cancer. The method includes delivering a cancer drug and a nuclease to a tumor. The cancer drug may be actinomycin, all-trans retinoic acid, anthracycline, bleomycin, bortezomib, carboplatin, carfilzomib, capecitabine, cisplatin, chlorambucil, cyclophosphamide, cytarabine, daunorubicin, disulfiram, docetaxel, doxifluridine, doxorubicin, epirubicin, epothilone, epoxomicin, etoposide, fluorouracil, gemcitabine, hydroxyurea, idarubicin, imatinib, interferon alpha, irinotecan, ixazomib, lactacystin, mechlorethamine, mercaptopurine, methotrexate, mitoxantrone, oxaliplatin, paclitaxel, pemetrexed, salinosporamide A, tenipo side, topotecan, valrubicin, vinblastine, vincristine, vindesine, and vinorelbine. The nuclease may be, for example, an endonuclease, an exonuclease, DNase I, a CRISPR-associated nuclease, Cfp1, a transcription-activator-like effector nuclease, a meganuclease, and a zinc-finger nuclease.

In certain embodiments, the nuclease preferentially cuts nucleic acid of a an oncovirus such as a human papilloma virus (HPV), an Epstein-Barr virus (EBV), Kaposi's sarcoma-associated herpesvirus (KSHV), hepatitis B virus (HBV), hepatitis C virus (HCV), and human T-cell lymphotrophic virus type I (HTLV-I). The nuclease may be a CRISPR-associated nuclease, and the composition further include a guide RNA complementary to a portion of the nucleic acid.

The cancer drug may be a proteasome inhibitor such as lactacystin, bortezomib, disulfiram, salinosporamide A, carfilzomib, epoxomicin, and ixazomib. In one embodiment, the nuclease is Cas9 and the oncovirus is Epstein-Barr virus. The cancer drug may be bortezomib.

The method may include delivering an antiviral treatment such as ganciclovir or Gardasil. The method may include delivering an epigenetic modifier such as a DNA methyltransferase (DNMT) inhibitor or a histone deacetylase inhibitor (HDI).

### BRIEF DESCRIPTION OF THE DRAWINGS

FIG. 1 diagrams a cancer treatment method.

FIG. 2 shows a cancer treatment composition.

FIG. 3 shows a composition that includes a nuclease and a chemotherapeutic.

FIG. 4 shows a plasmid that encodes the Cas9 protein.

FIG. 5 shows gRNA targets along a reference genome.

FIG. 6 depicts a proteasome inhibitor.

FIG. 7 illustrates gene delivery with an AAV vector.

FIG. 8 shows delivery by liposome.

FIG. 9 shows genomic context around guide RNA sgEBV2 and PCR primer locations.

FIG. 10 shows a large deletion induced by sgEBV2.

FIG. 11 gives genome context around guide RNA sgEBV3/4/5 and PCR primer locations.

FIG. 12 shows large deletions induced by Cas9.

FIG. 13 shows results confirmed by Sanger sequencing.

FIG. 14 shows several cell proliferation curves after different CRISPR treatments.

FIG. 15 shows nuclear morphology before sgEBV1-7 treatment.

FIG. 16 shows nuclear morphology after sgEBV1-7 treatment.

FIG. 17 shows EBV load after different CRISPR treatments.

FIG. 18 gives a histogram of EBV quantitative PCR Ct values before treatment.

FIG. 19 gives a histogram of EBV quantitative PCR Ct values after treatment.

### DETAILED DESCRIPTION

The invention provides compositions and methods for treating or preventing oncoviral infections and tumors. Compositions and methods according to the disclosure use a nuclease such as one that may be targeted to viral nucleic acid. For example, a Cas9 nuclease uses a targeting sequence, or guide RNA, to target the viral nucleic acid. The targeted cells are treated with the nuclease and a cancer drug. Each of those treatment modalities are introduced to the target cells. The nuclease cuts the viral nucleic acid and the cancer drug exhibits its chemotherapeutic effect. Either or both of the nuclease and cancer drug may be provided in a pharmaceutically acceptable composition.

i. Oncoviral Treatment

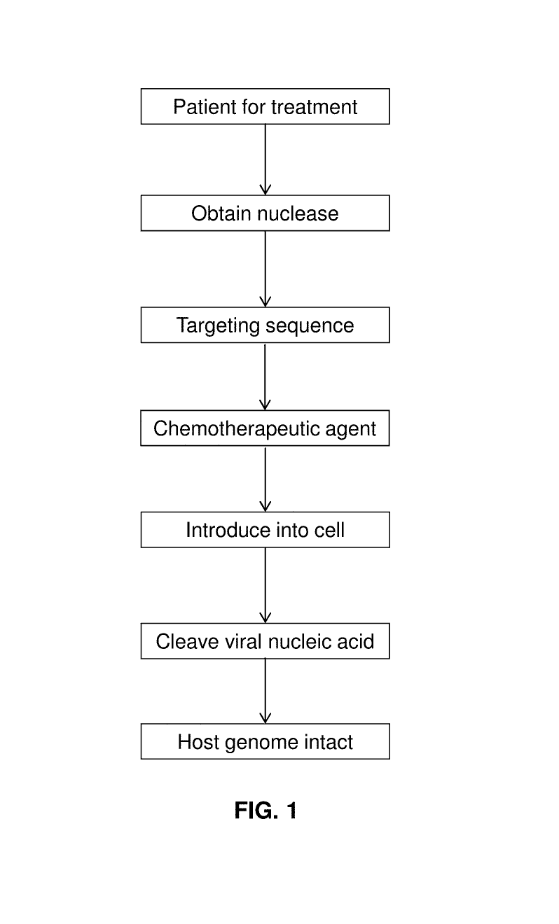

FIG. 1 diagrams a cancer treatment method that includes treating cells of a patient with a nuclease that cuts nucleic acid of an oncogenic virus and a cancer drug. Methods include obtaining a nuclease (e.g., as a protein, ribonucleoprotein, or encoded by a plasmid). Any suitable nuclease may be used. In a preferred embodiment, the nuclease is a CRISPR-associated nuclease or similar, such as Cas9, Cas6, a modified Cas9, a modified Cas6, Cfp1, or similar (collectively, “Cas-type nuclease”). Where a Cas-type nuclease is used, a targeting sequence is also used, where a targeting sequencing is an RNA oligomer, which may be about 20 bases long. In some embodiments, the nuclease comprises Cas9 complexed with a guide RNA complementary to a portion of the nucleic acid. Methods further include delivering a cancer drug. A cancer drug may be selected for any suitable mechanism of action including, for example, proteasome inhibition, transcription inhibition, inhibition of topoisomerase, chromatin remodeling action, inhibition of nucleotide synthesis, causation of DNA cross-linking, inhibition of DNA synthesis, affecting tubulin or microtubule binding, or others.

The nuclease (in active form or encoded in nucleic acid) are delivered to the cells of the patient. There, the nuclease cleaves nucleic acid of the oncovirus. For example, the nuclease may cleave DNA or RNA genome products (e.g., episomal, integrated, or otherwise) or may cleave transcripts. Through the use of the targeting sequence, the nuclease leaves intact important portions of the host genome necessary for healthy function. The cancer drug aids in treating or preventing cell proliferation through its preferred mechanism of action.

Methods of the invention are applicable to in vivo treatment of patients and may be used to remove any viral genetic material such as genes of virus associated with a latent viral infection. Methods may be used in vitro, e.g., to prepare or treat a cell culture or cell sample. When used in vivo, the cell may be any suitable germ line or somatic cell and compositions of the invention may be delivered to specific parts of a patient's body or be delivered systemically. If delivered systemically, it may be preferable to include within compositions of the invention tissue-specific promoters. For example, if a patient has a latent viral infection that is localized to the liver, hepatic tissue-specific promoters may be included in a plasmid or viral vector that codes for a targeted nuclease.

Any suitable oncovirus may be targeted using methods and compositions of the invention. For example, a human papilloma virus (HPV), an Epstein-Barr virus (EBV), Kaposi's sarcoma-associated herpesvirus (KSHV), hepatitis B virus (HBV), hepatitis C virus (HCV), human T-cell lymphotrophic virus type I (HTLV-I), or Merkel cell polyomavirus (MCV) may be treated.

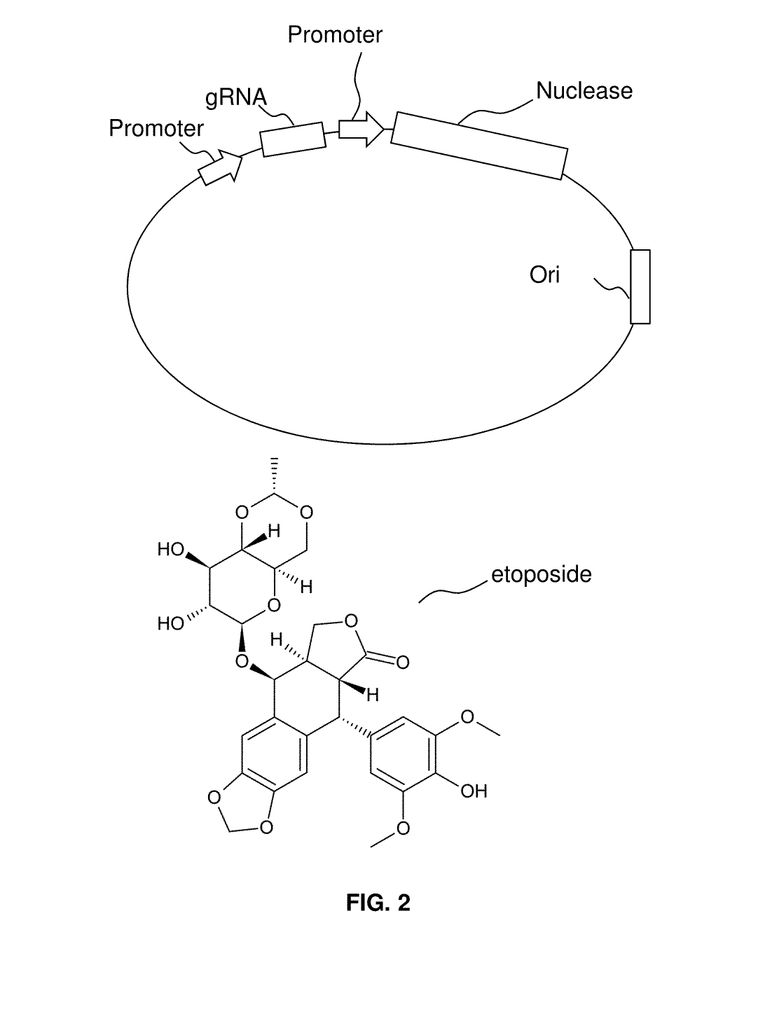

FIG. 2 shows a composition for treating a viral infection according to certain embodiments. The composition preferably includes a vector (which may be a plasmid, linear DNA, or a viral vector) that codes for a nuclease and a targeting moiety (e.g., a gRNA) that targets the nuclease to viral nucleic acid and a chemotherapeutic such as etoposide. The vector may optionally include one or more of a promoter, replication origin, other elements, or combinations thereof as described further herein.

In some embodiments, the invention provides a nucleic acid encoding at least (i) a Cas9 nuclease and (ii) a guide RNA (gRNA) complementary to a portion of the Epstein-Barr genome as well as a chemotherapeutic such as etoposide, preferably all of the components of EPOCH with rituximab (rituximab, etoposide, prednisolone, oncovin: vincristine, cyclophosphamide, and hydroxydaunorubicin: doxorubicin. In a related embodiment, what is provided includes (i) a Cas9 nuclease and (ii) a guide RNA (gRNA) complementary to a portion of the Epstein-Barr genome as well as a chemotherapeutic such as etoposide, preferably all of the components of EPOCH with rituximab (rituximab, etoposide, prednisolone, oncovin: vincristine, cyclophosphamide, and hydroxydaunorubicin: doxorubicin.

ii. Nuclease

Methods of the invention include using a programmable or targetable nuclease to specifically target viral nucleic acid for destruction. Any suitable targeting nuclease can be used including, for example, zinc-finger nucleases (ZFNs), transcription activator-like effector nucleases (TALENs), clustered regularly interspaced short palindromic repeat (CRISPR) nucleases, meganucleases, other endo- or exo-nucleases, or combinations thereof. See Schiffer, 2012, Targeted DNA mutagenesis for the cure of chronic viral infections, J Virol 88(17):8920-8936, incorporated by reference.

CRISPR methodologies employ a nuclease, CRISPR-associated (Cas9), that complexes with small RNAs as guides (gRNAs) to cleave DNA in a sequence-specific manner upstream of the protospacer adjacent motif (PAM) in any genomic location. CRISPR may use separate guide RNAs known as the crRNA and tracrRNA. These two separate RNAs have been combined into a single RNA to enable site-specific mammalian genome cutting through the design of a short guide RNA. Cas9 and guide RNA (gRNA) may be synthesized by known methods. Cas9/guide-RNA (gRNA) uses a non-specific DNA cleavage protein Cas9, and an RNA oligo to hybridize to target and recruit the Cas9/gRNA complex. See Chang et al., 2013, Genome editing with RNA-guided Cas9 nuclease in zebrafish embryos, Cell Res 23:465-472; Hwang et al., 2013, Efficient genome editing in zebrafish using a CRISPR-Cas system, Nat. Biotechnol 31:227-229; Xiao et al., 2013, Chromosomal deletions and inversions mediated by TALENS and CRISPR/Cas in zebrafish, Nucl Acids Res 1-11.

CRISPR (Clustered Regularly Interspaced Short Palindromic Repeats) is found in bacteria and is believed to protect the bacteria from phage infection. It has been used to introduce insertions or deletions as a way of increasing or decreasing transcription in the DNA of a targeted cell or population of cells. See for example, Horvath et al., Science (2010) 327:167-170; Terns et al., Current Opinion in Microbiology (2011) 14:321-327; Bhaya et al. Ann Rev Genet (2011) 45:273-297; Wiedenheft et al. Nature (2012) 482:331-338); Jinek Met al. Science (2012) 337:816-821; Cong Let al. Science (2013) 339:819-823; Jinek M et al. (2013) eLife 2:e00471; Mali Pet al. (2013) Science 339:823-826; Qi LS et al. (2013) Cell 152:1173-1183; Gilbert LA et al. (2013) Cell 154:442-451; Yang H et al. (2013) Cell 154:1370-1379; and Wang H et al. (2013) Cell 153:910-918).

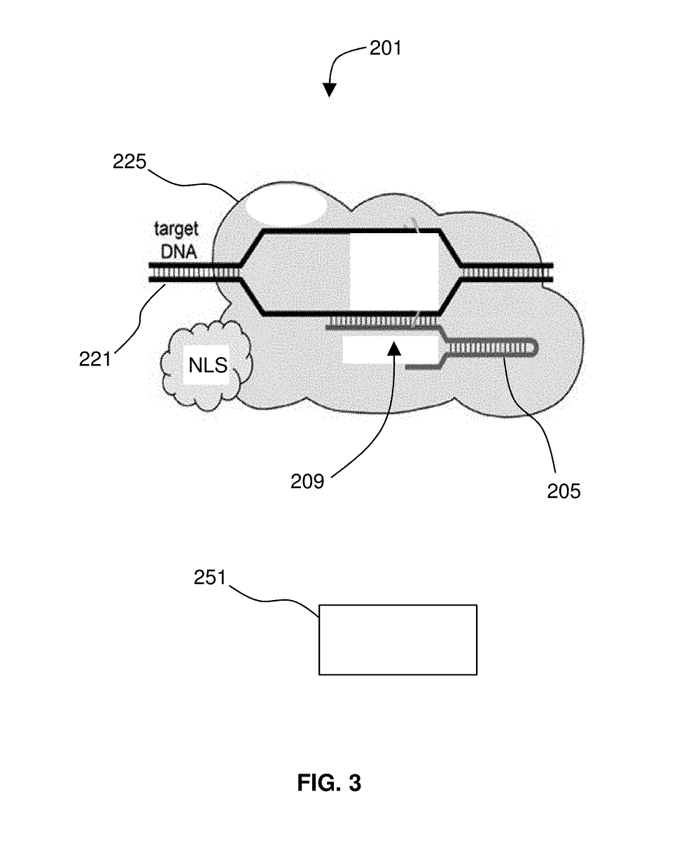

FIG. 3 shows a nuclease 201 and a cancer drug 251. Here, the nuclease 201 is illustrated as a ribonucleoprotein (RNP) that includes a Cas9/gRNA complex. The Cas9/gRNA complex includes a Cas9 endonuclease 225 in a complex with a single guide RNA (sgRNA) 205, bound to the target 221 oncoviral nucleic acid via the guide sequence 209 of the guide RNA. The target 221 included to aid in understanding. Compositions of the invention according to some embodiments include the RNP (which provides the nuclease 201) and the cancer drug 251. In other embodiments, the nuclease may be delivered in the form of a protein or a nucleic acid (e.g., as mRNA or encoded on a plasmid). The cancer drug 251 may be any suitable agent such as one of those discussed below.

In an aspect of the invention, the Cas9 endonuclease causes a double strand break in at least two locations in oncoviral nucleic acid. These two double strand breaks cause a fragment to be deleted. Even if viral repair pathways anneal the two ends, there will still be a deletion in the genome. One or more deletions using the mechanism will incapacitate the virus. The result is that the host cell will be free of viral infection.

In embodiments of the invention, nucleases cleave the genome of the target virus. A nuclease is an enzyme capable of cleaving the phosphodiester bonds between the nucleotide subunits of nucleic acids. Endonucleases are enzymes that cleave the phosphodiester bond within a polynucleotide chain. Some, such as deoxy-ribonuclease I, cut DNA relatively nonspecifically (without regard to sequence), while many, typically called restriction endonucleases or restriction enzymes, cleave only at very specific nucleotide sequences. In a preferred embodiment of the invention, the Cas9 nuclease is incorporated into the compositions and methods of the invention, however, it should be appreciated that any nuclease may be used.

In preferred embodiments of the invention, the Cas9 nuclease is used to cleave the genome. The Cas9 nuclease is capable of creating a double strand break in the genome. The Cas9 nuclease has two functional domains: RuvC and HNH, each cutting a different strand. When both of these domains are active, the Cas9 causes double strand breaks in the genome.

In some embodiments of the invention, insertions into the genome can be designed to cause incapacitation, or altered genomic expression. Additionally, insertions/deletions are also used to introduce a premature stop codon either by creating one at the double strand break or by shifting the reading frame to create one downstream of the double strand break. Any of these outcomes of the NHEJ repair pathway can be leveraged to disrupt the target gene. The changes introduced by the use of the CRISPR/gRNA/Cas9 system are permanent to the genome.

In some embodiments of the invention, at least one insertion is caused by the CRISPR/gRNA/Cas9 complex. In a preferred embodiment, numerous insertions are caused in the genome, thereby incapacitating the virus. In an aspect of the invention, the number of insertions lowers the probability that the genome may be repaired.

In some embodiments of the invention, at least one deletion is caused by Cas9. In a preferred embodiment, numerous deletions are caused in the genome, thereby incapacitating the virus. In an aspect of the invention, the number of deletions lowers the probability that the genome may be repaired. In a highly-preferred embodiment, Cas9 causes significant genomic disruption, resulting in effective destruction of the viral genome, while leaving the host genome intact. It is noted that in treating a tumor or other oncoviral infection, repair of cleaved DNA by host ligases (e.g., by non-homologous end joining) may not be required. The absence of host-mediated repair may be an aid in disrupting viral or tumor DNA and may aid in inducing cell death.

TALENs uses a nonspecific DNA-cleaving nuclease fused to a DNA-binding domain that can be to target essentially any sequence. For TALEN technology, target sites are identified and expression vectors are made. Linearized expression vectors (e.g., by Not1) may be used as template for mRNA synthesis. A commercially available kit may be use such as the mMESSAGE mMACHINE SP6 transcription kit from Life Technologies (Carlsbad, Calif.). See Joung & Sander, 2013, TALENs: a widely applicable technology for targeted genome editing, Nat Rev Mol Cell Bio 14:49-55.

TALENs and CRISPR methods provide one-to-one relationship to the target sites, i.e. one unit of the tandem repeat in the TALE domain recognizes one nucleotide in the target site, and the crRNA, gRNA, or sgRNA of CRISPR/Cas system hybridizes to the complementary sequence in the DNA target. Methods can include using a pair of TALENs or a Cas9 protein with one gRNA to generate double-strand breaks in the target. The breaks are then repaired via non-homologous end-joining or homologous recombination (HR).

ZFN may be used to cut viral nucleic acid. Briefly, the ZFN method includes introducing into the infected host cell at least one vector (e.g., RNA molecule) encoding a targeted ZFN 305 and, optionally, at least one accessory polynucleotide. See, e.g., U.S. Pub. 2011/0023144 to Weinstein, incorporated by reference The cell includes target sequence. The cell is incubated to allow expression of the ZFN, wherein a double-stranded break is introduced into the targeted chromosomal sequence by the ZFN. In some embodiments, a donor polynucleotide or exchange polynucleotide is introduced. Swapping a portion of the viral nucleic acid with irrelevant sequence can fully interfere transcription or replication of the viral nucleic acid. Target DNA along with exchange polynucleotide may be repaired by an error-prone non-homologous end-joining DNA repair process or a homology-directed DNA repair process.

Typically, a ZFN comprises a DNA binding domain (i.e., zinc finger) and a cleavage domain (i.e., nuclease) and this gene may be introduced as mRNA (e.g., 5′ capped, polyadenylated, or both). Zinc finger binding domains may be engineered to recognize and bind to any nucleic acid sequence of choice. See, e.g., Qu et al., 2013, Zinc-finger-nucleases mediate specific and efficient excision of HIV-1 proviral DAN from infected and latently infected human T cells, Nucl Ac Res 41(16):7771-7782, incorporated by reference. An engineered zinc finger binding domain may have a novel binding specificity compared to a naturally-occurring zinc finger protein. Engineering methods include, but are not limited to, rational design and various types of selection. A zinc finger binding domain may be designed to recognize a target DNA sequence via zinc finger recognition regions (i.e., zinc fingers). See for example, U.S. Pat. Nos. 6,607,882; 6,534,261 and 6,453,242, incorporated by reference. Exemplary methods of selecting a zinc finger recognition region may include phage display and two-hybrid systems, and are disclosed in U.S. Pat. No. 5,789,538; U.S. Pat. No. 5,925,523; U.S. Pat. No. 6,007,988; U.S. Pat. No. 6,013,453; U.S. Pat. No. 6,410,248; U.S. Pat. No. 6,140,466; U.S. Pat. No. 6,200,759; and U.S. Pat. No. 6,242,568, each of which is incorporated by reference.

A ZFN also includes a cleavage domain. The cleavage domain portion of the ZFNs may be obtained from any suitable endonuclease or exonuclease such as restriction endonucleases and homing endonucleases. See, for example, Belfort & Roberts, 1997, Homing endonucleases: keeping the house in order, Nucleic Acids Res 25(17):3379-3388. A cleavage domain may be derived from an enzyme that requires dimerization for cleavage activity. Two ZFNs may be required for cleavage, as each nuclease comprises a monomer of the active enzyme dimer. Alternatively, a single ZFN may comprise both monomers to create an active enzyme dimer. Restriction endonucleases present may be capable of sequence-specific binding and cleavage of DNA at or near the site of binding. Certain restriction enzymes (e.g., Type IIS) cleave DNA at sites removed from the recognition site and have separable binding and cleavage domains. For example, the Type IIS enzyme FokI, active as a dimer, catalyzes double-stranded cleavage of DNA, at 9 nucleotides from its recognition site on one strand and 13 nucleotides from its recognition site on the other. The FokI enzyme used in a ZFN may be considered a cleavage monomer. Thus, for targeted double-stranded cleavage using a FokI cleavage domain, two ZFNs, each comprising a FokI cleavage monomer, may be used to reconstitute an active enzyme dimer. See Wah, et al., 1998, Structure of FokI has implications for DNA cleavage, PNAS 95:10564-10569; U.S. Pat. No. 5,356,802; U.S. Pat. No. 5,436,150; U.S. Pat. No. 5,487,994; U.S. Pub. 2005/0064474; U.S. Pub. 2006/0188987; and U.S. Pub. 2008/0131962, each incorporated by reference.

Virus targeting using ZFN may include introducing at least one donor polynucleotide comprising a sequence into the cell. A donor polynucleotide preferably includes the sequence to be introduced flanked by an upstream and downstream sequence that share sequence similarity with either side of the site of integration in the chromosome. The upstream and downstream sequences in the donor polynucleotide are selected to promote recombination between the chromosomal sequence of interest and the donor polynucleotide. Typically, the donor polynucleotide will be DNA. The donor polynucleotide may be a DNA plasmid, a bacterial artificial chromosome (BAC), a yeast artificial chromosome (YAC), a viral vector, a linear piece of DNA, a PCR fragment, a naked nucleic acid, and may employ a delivery vehicle such as a liposome. The sequence of the donor polynucleotide may include exons, introns, regulatory sequences, or combinations thereof. The double stranded break is repaired via homologous recombination with the donor polynucleotide such that the desired sequence is integrated into the chromosome. In the ZFN-mediated process, a double stranded break introduced into the target sequence by the ZFN is repaired, via homologous recombination with the exchange polynucleotide, such that the sequence in the exchange polynucleotide may be exchanged with a portion of the target sequence. The presence of the double stranded break facilitates homologous recombination and repair of the break. The exchange polynucleotide may be physically integrated or, alternatively, the exchange polynucleotide may be used as a template for repair of the break, resulting in the exchange of the sequence information in the exchange polynucleotide with the sequence information in that portion of the target sequence. Thus, a portion of the viral nucleic acid may be converted to the sequence of the exchange polynucleotide. ZFN methods can include using a vector to deliver a nucleic acid molecule encoding a ZFN and, optionally, at least one exchange polynucleotide or at least one donor polynucleotide to the infected cell.

Meganucleases are endo-deoxy-ribonucleases characterized by a large recognition site (double-stranded DNA sequences of 12 to 40 base pairs); as a result this site generally occurs only once in any given genome. For example, the 18-base pair sequence recognized by the I-SceI meganuclease would on average require a genome twenty times the size of the human genome to be found once by chance (although sequences with a single mismatch occur about three times per human-sized genome). Meganucleases are therefore considered to be the most specific naturally occurring restriction enzymes. Meganucleases can be divided into five families based on sequence and structure motifs. See, e.g., U.S. Pub. 2010/0086533; U.S. Pub. 2014/0208457; and Silva et al., 2011, Meganucleases and other tools for targeted genome engineering, Cur Gene Ther 11(1):11-27, the contents of each of which are incorporated by reference.

In some embodiments of the invention, a template sequence is inserted into the genome. In order to introduce nucleotide modifications to genomic DNA, a DNA repair template containing the desired sequence must be present during homology directed repair (HDR). The DNA template is normally transfected into the cell along with the gRNA/Cas9. The length and binding position of each homology arm is dependent on the size of the change being introduced. In the presence of a suitable template, HDR can introduce significant changes at the Cas9 induced double strand break.

Some embodiments of the invention may utilize modified version of a nuclease. Modified versions of the Cas9 enzyme containing a single inactive catalytic domain, either RuvC- or HNH-, are called ‘nickases’. With only one active nuclease domain, the Cas9 nickase cuts only one strand of the target DNA, creating a single-strand break or ‘nick’. Similar to the inactive dCas9 (RuvC- and HNH-), a Cas9 nickase is still able to bind DNA based on gRNA specificity, though nickases will only cut one of the DNA strands. The majority of CRISPR plasmids are derived from S. pyogenes and the RuvC domain can be inactivated by a D10A mutation and the HNH domain can be inactivated by an H840A mutation.

A single-strand break, or nick, is normally quickly repaired through the HDR pathway, using the intact complementary DNA strand as the template. However, two proximal, opposite strand nicks introduced by a Cas9 nickase are treated as a double strand break, in what is often referred to as a ‘double nick’ or ‘dual nickase’ CRISPR system. A double-nick induced double strain break can be repaired by either NHEJ or HDR depending on the desired effect on the gene target. At these double strain breaks, insertions and deletions are caused by the CRISPR/Cas9 complex. In an aspect of the invention, a deletion is caused by positioning two double strand breaks proximate to one another, thereby causing a fragment of the genome to be deleted.

iii. Targeting sequence

A nuclease may use the targeting specificity of a guide RNA (gRNA). As discussed below, guide RNAs or single guide RNAs are specifically designed to target a virus genome. As used herein targeting sequence can mean any combination of gRNA, crRNA, tracrRNA, sgRNA, and others. A CRISPR/Cas9 gene editing complex of the invention works optimally with a guide RNA that targets the viral genome. Guide RNA (gRNA) (which includes single guide RNA (sgRNA), crisprRNA (crRNA), trans-activating RNA (tracrRNA), any other targeting oligo, or any combination thereof) leads the CRISPR/Cas9 complex to the viral genome in order to cause viral genomic disruption. In an aspect of the invention, CRISPR/Cas9/gRNA complexes are designed to target specific viruses within a cell. It should be appreciated that any virus can be targeted using the composition of the invention. Identification of specific regions of the virus genome aids in development and designing of CRISPR/Cas9/gRNA complexes.

In an aspect of the invention, the CRISPR/Cas9/gRNA complexes are designed to target latent viruses within a cell. Once transfected within a cell, the CRISPR/Cas9/gRNA complexes cause repeated insertions or deletions to render the genome incapacitated, or due to number of insertions or deletions, the probability of repair is significantly reduced.

As an example, the Epstein-Barr virus (EBV), also called human herpesvirus 4 (HHV-4), is inactivated in cells using a CRISPR/Cas9/gRNA complex. EBV is a virus of the herpes family, and is one of the most common viruses in humans. The virus is approximately 122 nm to 180 nm in diameter and is composed of a double helix of DNA wrapped in a protein capsid. In this example, the Raji cell line serves as an appropriate in vitro model. The Raji cell line is the first continuous human cell line from hematopoietic origin and cell lines produce an unusual strain of Epstein-Barr virus while being one of the most extensively studied EBV models. To target the EBV genomes in the Raji cells, a CRISPR/Cas9 complex with specificity for EBV is needed.

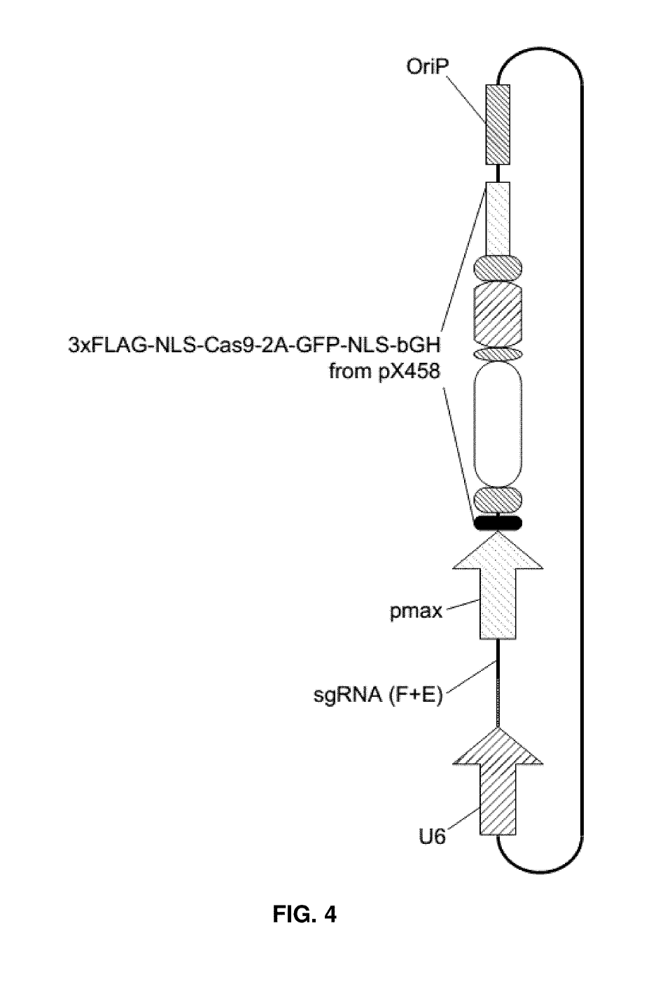

FIG. 4 shows a plasmid that includes an EGFP marker fused after the Cas9 protein.

The design of EBV-targeting CRISPR/Cas9 plasmids consisting of a U6 promoter driven chimeric guide RNA (sgRNA) and a ubiquitous promoter driven Cas9 that were obtained from Addgene, Inc. Commercially available guide RNAs and Cas9 nucleases may be used with the present invention. The EGFP marker fused after the Cas9 protein allowed selection of Cas9-positive cells.

Preferably guide RNAs are designed, whether or not commercially purchased, to target a specific part of an HPV genome. The target area in HPV is identified and guide RNA to target selected portions of the HPV genome are developed and incorporated into the composition of the invention. In an aspect of the invention, a reference genome of a particular strain of the virus is selected for guide RNA design.

In relation to EBV, for example, the reference genome from strain B95-8 was used as a design guide. Within a genome of interest, such as EBV, selected regions, or genes are targeted. For example, six regions can be targeted with seven guide RNA designs for different genome editing purposes.

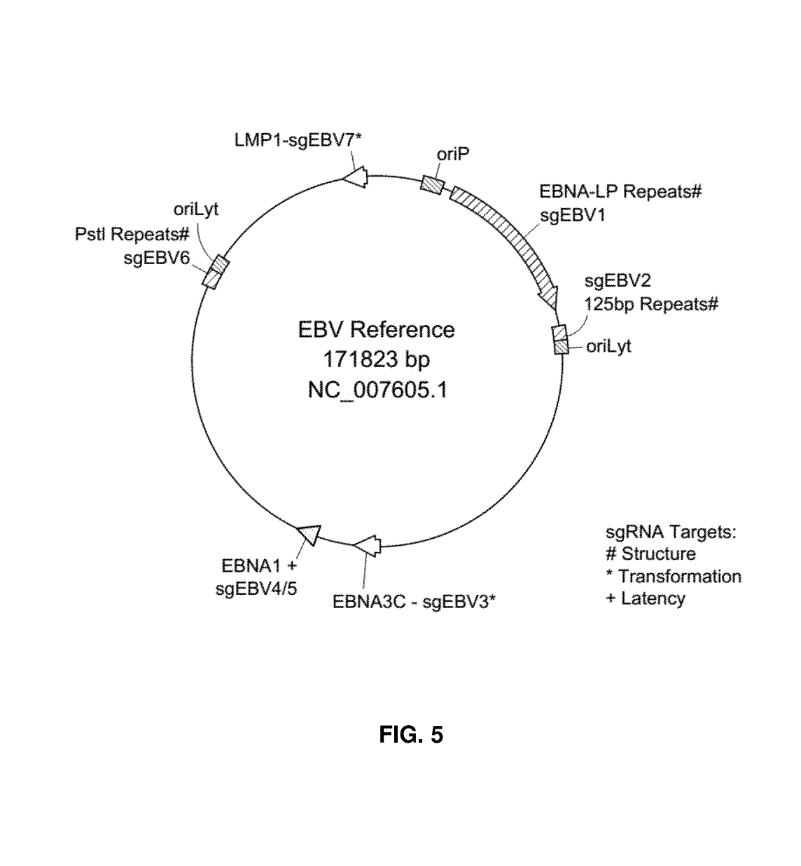

FIG. 5 shows gRNA targets along a reference genome where # denotes structural targets, where * denotes transformation-related targets, and where + denotes latency-related targets.

In relation to EBV, EBNA1 is the only nuclear Epstein-Barr virus (EBV) protein expressed in both latent and lytic modes of infection. While EBNA1 is known to play several important roles in latent infection, EBNA1 is crucial for many EBV functions including gene regulation and latent genome replication. Therefore, guide RNAs sgEBV4 and sgEBV5 were selected to target both ends of the EBNA1 coding region in order to excise this whole region of the genome. These “structural” targets enable systematic digestion of the EBV genome into smaller pieces. EBNA3C and LMP1 are essential for host cell transformation, and guide RNAs sgEBV3 and sgEBV7 were designed to target the 5′ exons of these two proteins respectively.

In certain embodiments, the invention uses Cas9 or another Cas-type nuclease (Cas6, Cfp1, modified Cas9, modified Cas6, modified Cfp1, etc.) with one or a plurality of guide RNAs with a sequence specific to a target in a genome of Merkel cell polyomavirus (MCV), delivered in conjunction with a cancer therapeutic. Merkel cell polyomavirus (MCV), which can cause merkel cell carcinoma (MCV). MCV is the fifth polyomavirus that infects humans to be discovered. Polyomaviruses are small (˜5400 base pair), non-enveloped, double-stranded DNA viruses. MCV is one of seven currently known human oncoviruses. It is suspected to cause the majority of cases of Merkel cell carcinoma, a rare but aggressive form of skin cancer.

iv. Cancer Drug

Methods and compositions of the invention use one or a plurality of cancer drug in conjunction with a nuclease to treat or prevent an oncoviral infection or resultant condition. The cancer drug(s) may be selected for its mechanism of action, its clinical effectiveness, its suitability to a particular cancer of infectious origin, or any other suitable trait. When delivered to a patient, the agent will have an effect according to its mechanism of action.

It may be preferable to use a proteasome inhibitor. Proteasome inhibitors are drugs that block the action of proteasomes, cellular complexes that break down proteins. Multiple mechanisms are likely to be involved, but proteasome inhibition may prevent degradation of pro-apoptotic factors such as the p53 protein, permitting activation of programmed cell death in neoplastic cells dependent upon suppression of pro-apoptotic pathways. For example, bortezomib causes a rapid and dramatic change in the levels of intracellular peptides. Suitable proteasome inhibitors may include bortezomib, lactacystin, disulfiram, Salinosporamide A, bortezomib, carfilzomib, epoxomicin, and ixazomib.

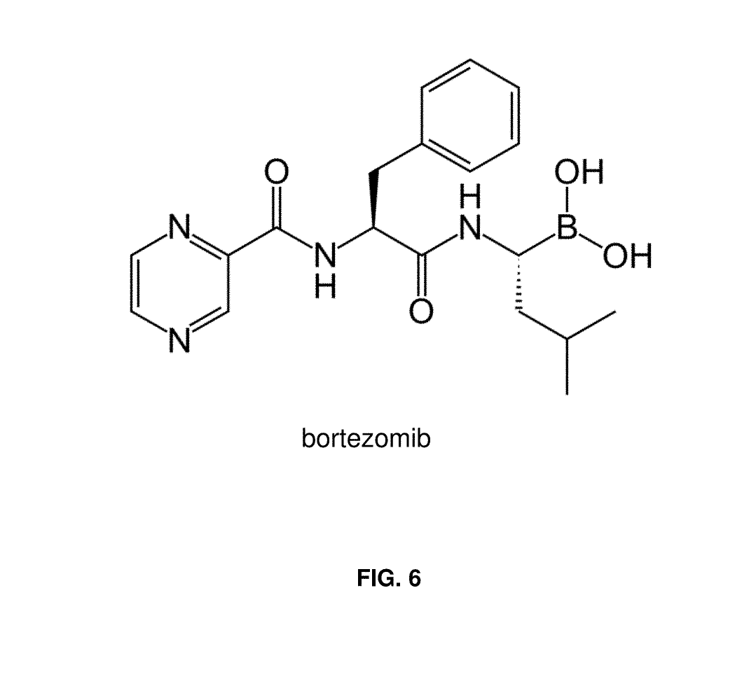

FIG. 6 depicts the proteasome inhibitor bortezomib. A boron atom in bortezomib binds the catalytic site of the 26S proteasome with high affinity and specificity. In normal cells, the proteasome regulates protein expression and function by degradation of ubiquitylated proteins, and also cleanses the cell of abnormal or misfolded proteins. Clinical and preclinical data support a role in maintaining the immortal phenotype of myeloma cells, and cell-culture and xenograft data support a similar function in solid tumor cancers. While multiple mechanisms are likely to be involved, proteasome inhibition may prevent degradation of pro-apoptotic factors, permitting activation of programmed cell death in neoplastic cells dependent upon suppression of pro-apoptotic pathways.

Lactacystin binds and inhibits specific catalytic subunits of the proteasome, a protein complex responsible for the bulk of proteolysis in the cell, as well as proteolytic activation of certain protein substrates. Lactacystin covalently modifies the amino-terminal threonine of specific catalytic subunits of the proteasome

Disulfiram creates complexes with metals (dithiocarbamate complexes) and acts as a proteasome inhibitor. A clinical trial of disulfiram with copper gluconate against liver cancer is being conducted in Utah and a clinical trial of disulfiram as adjuvant against lung cancer is happening in Israel.

Salinosporamide A is a potent proteasome inhibitor and potential anticancer agent. Salinosporamide A inhibits proteasome activity by covalently modifying the active site threonine residues of the 20S proteasome. In vitro studies using purified 20S proteasomes showed that salinosporamide A has lower EC50 for trypsin-like (T-L) activity than does bortezomib. In vivo animal model studies show marked inhibition of T-L activity in response to salinosporamide A, whereas bortezomib enhances T-L proteasome activity.

Carfilzomib (marketed under the trade name Kyprolis (Onyx Pharmaceuticals, Inc.) is an anti-cancer drug acting as a selective proteasome inhibitor. Chemically, carfilzomib is a tetrapeptide epoxyketone and an analog of epoxomicin. Carfilzomib irreversibly binds to and inhibits the chymotrypsin-like activity of the 20S proteasome, an enzyme that degrades unwanted cellular proteins. Inhibition of proteasome-mediated proteolysis results in a build-up of poly-ubiquinated proteins, which may cause cell cycle arrest, apoptosis, and inhibition of tumor growth.

Epoxomicin is a naturally occurring selective proteasome inhibitor with anti-inflammatory activity.

Ixazomib is a proteasome inhibitor similar to bortezomib. Ixazomib is considered to be a second-generation proteasome inhibitor because it has improved characteristics and activity over Velcade.

A cancer drug may be selected for any suitable mechanism of action including, for example, transcription inhibition, inhibition of topoisomerase, chromatin remodeling action, inhibition of nucleotide synthesis, causation of DNA cross-linking, inhibition of DNA synthesis, affecting tubulin or microtubule binding, or others. These categories may overlap and may not be mutually exclusive. An exemplary transcription inhibitor includes actinomycin D. Suitable topoisomerase inhibitors include idarubicin, irinotecan, topotecan, mitoxantrone, and daunorubicin. In some embodiments, the cancer drug contributes to chromatin remodeling or to the breakage of nucleic acid strands. For example, bleomycin and teniposide are known to cause breaks in DNA strands. Suitable nucleotide synthesis inhibitors include capecitabine, hydroxycarbamide and pemetrexed. Suitable DNA cross-linkers include cisplatin, mechlorethamine, and oxaliplatin. Cancer drugs that inhibit DNA synthesis include, e.g., chlorambucil, gemcitabine, capecitabine, and cytarabine. Cancer drugs that affect tubulin/microtubule binding include, e.g., docetaxel, paclitaxel, vinblastine, vincristine, and vinorelbine. By delivery of the cancer drug, cell proliferation is inhibited and the growth of any tumor may be suppressed. Thus the adverse effects of infection by an oncovirus may be minimized. For example, methods of the invention may be used to treat children living in areas associated with a high prevalence of Burkitt's lymphoma. Such a patient may be treated with a nuclease that specifically cuts nucleic acid of the Epstein-Barr virus—without hindering the normal, healthy function of the human genome—and an anti-tumor cancer drug to prevent or treat a Burkitt's lymphoma.

Exemplary cancer drugs that may be used include the following.

Actinomycin D is the most significant member of actinomycines, which are a class of polypeptide antitumor antibiotics isolated from soil bacteria of the genus Streptomyces. Actinomycin D is one of the older anticancer drugs, and has been used for many years. Actinomycin D is shown to have the ability to inhibit transcription. Actinomycin D does this by binding DNA at the transcription initiation complex and preventing elongation of RNA chain by RNA polymerase.

Tretinoin, also known as all-trans retinoic acid, is used to treat at least one form of cancer (acute promyelocytic leukemia, also called acute myeloid leukemia subtype M3) by causing the immature promyelocytes to differentiate (i.e. mature). The pathology of the leukemia is due to the highly proliferative immature cells; retinoic acid drives these cells to develop into functional cells, which helps to alleviate the disease.

Anthracyclines (e.g., daunorubicin) are a class of drugs (CCNS or cell-cycle non-specific) used in cancer chemotherapy derived from Streptomyces bacterium. Anthracyclines are used to treat many cancers, including leukemias, lymphomas, breast, stomach, uterine, ovarian, bladder cancer, and lung cancers. Anthracyclines have four mechanisms of action: inhibition of DNA and RNA synthesis; inhibition of topoisomerase II enzyme; iron-mediated generation of free oxygen radicals; and induction of histone eviction from chromatin.

Bleomycin acts by induction of DNA strand breaks. Some studies suggest bleomycin also inhibits incorporation of thymidine into DNA strands. DNA cleavage by bleomycin depends on oxygen and metal ions, at least in vitro. The exact mechanism of DNA strand scission is unresolved, but it has been suggested that bleomycin chelates metal ions (primarily iron), producing a pseudo-enzyme that reacts with oxygen to produce superoxide and hydroxide free radicals that cleave DNA.

Carboplatin is a chemotherapy drug used against some forms of cancer.

Capecitabine is a cancer drug used in the treatment of numerous cancers. Capecitabine is metabolized to 5-FU which in turn is a thymidylate synthase inhibitor, hence inhibiting the synthesis of thymidine monophosphate (TMP), the active form of thymidine which is required for the de novo synthesis of DNA.

Cisplatin is a chemotherapy drug, a member of a class of platinum-containing anti-cancer drugs, which now also includes carboplatin and oxaliplatin. These platinum complexes react in vivo, binding to and causing crosslinking of DNA, which ultimately triggers apoptosis (programmed cell death).

Chlorambucil is a chemotherapy drug that has been mainly used in the treatment of chronic lymphocytic leukemia. It is a nitrogen mustard alkylating agent and can be given orally. Chlorambucil produces its anti-cancer effects by interfering with DNA replication and damaging the DNA in a cell. The DNA damage induces cell cycle arrest and cellular apoptosis via the accumulation of cytosolic p53 and subsequent activation of Bax, an apoptosis promoter. Chlorambucil alkylates and cross-links DNA during all phases of the cell cycle, inducing DNA damage via three different methods of covalent adduct generation with double-helical DNA

Cyclophosphamide is metabolized to phosphoramide mustard. This metabolite is only formed in cells that have low levels of ALDH. Phosphoramide mustard forms DNA crosslinks both between and within DNA strands at guanine N-7 positions (known as inter-strand and intra-strand cross-linkages, respectively). This is irreversible and leads to cell apoptosis. Cyclophosphamide has relatively little typical chemotherapy toxicity as ALDHs are present in relatively large concentrations in bone marrow stem cells, liver and intestinal epithelium. ALDHs protect these actively proliferating tissues against toxic effects of phosphoramide mustard and acrolein by converting aldophosphamide to carboxycyclophosphamide that does not give rise to the toxic metabolites phosphoramide mustard and acrolein. This is because carboxycyclophosphamide cannot undergo β-elimination (the carboxylate acts as an electron-donating group, forbidding the transformation), preventing nitrogen mustard activation and subsequent alkylation.

Cytarabine is a cancer drug that interferes with the synthesis of DNA. It is an antimetabolic agent with the chemical name of 1β-arabinofuranosylcytosine. Its mode of action is due to its rapid conversion into cytosine arabinoside triphosphate, which damages DNA when the cell cycle holds in the S phase (synthesis of DNA). Rapidly dividing cells, which require DNA replication for mitosis, are therefore most affected. Cytosine arabinoside also inhibits both DNA and RNA polymerases and nucleotide reductase enzymes needed for DNA synthesis.

Daunorubicin, or daunomycin, is chemotherapeutic of the anthracycline family that interacts with DNA by intercalation and inhibition of macromolecular biosynthesis. This inhibits the progression of the enzyme topoisomerase II, which relaxes supercoils in DNA for transcription. Daunorubicin stabilizes the topoisomerase II complex after it has broken the DNA chain for replication, preventing the DNA double helix from being resealed and thereby stopping the process of replication. On binding to DNA, daunorubicin intercalates, with its daunosamine residue directed toward the minor groove. It can also induce histone eviction from chromatin upon intercalation.

Docetaxel is a chemotherapy medication that works by interfering with cell division. Docetaxel binds to microtubules reversibly with high affinity and has a maximum stoichiometry of 1 mole docetaxel per mole tubulin in microtubules. This binding stabilizes microtubules and prevents de-polymerization from calcium ions, decreased temperature and dilution, preferentially at the plus end of the microtubule. Docetaxel has been found to accumulate to higher concentration in ovarian adenocarcinoma cells than kidney carcinoma cells, which may contribute to the more effective treatment of ovarian cancer by docetaxel. It has also been found to lead to the phosphorylation of oncoprotein bcl-2, which is apoptosis-blocking in its oncoprotein form.

Doxifluridine is a fluoropyrimidine derivative and oral prodrug of the antineoplastic agent 5-fluorouracil (5-FU) with antitumor activity. Doxifluridine, designed to circumvent the rapid degradation of 5-FU by dihydropyrimidine dehydrogenase in the gut wall, is converted into 5-FU in the presence of pyrimidine nucleoside phosphorylase. 5-FU interferes with DNA synthesis and subsequent cell division by reducing normal thymidine production and interferes with RNA transcription by competing with uridine triphosphate for incorporation into the RNA strand.

Doxorubicin is an anthracycline antitumor antibiotic that works by intercalating DNA.

Epirubicin is an anthracycline drug used for chemotherapy that works by intercalating DNA strands. Intercalation results in complex formation which inhibits DNA and RNA synthesis. It also triggers DNA cleavage by topoisomerase II, resulting in mechanisms that lead to cell death.

The epothilones are a class of potential cancer drugs that prevent cancer cells from dividing by interfering with tubulin.

Fluorouracil (5-FU) sold as Adrucil among others, is a drug that is a pyrimidine analog which is used in the treatment of cancer. It is a suicide inhibitor and works through irreversible inhibition of thymidylate synthase.

Gemcitabine is a nucleoside analog used as chemotherapy. The triphosphate analogue of gemcitabine replaces one of the building blocks of nucleic acids, in this case cytidine, during DNA replication. The process arrests tumor growth, as only one additional nucleoside can be attached to the “faulty” nucleoside, resulting in apoptosis.

Hydroxycarbamide is an antineoplastic drug used in myeloproliferative disorders. Hydroxycarbamide decreases the production of deoxyribonucleotides via inhibition of the enzyme ribonucleotide reductase by scavenging tyrosyl free radicals as they are involved in the reduction NDPs.

Idarubicin is an anthracycline antileukemic drug that inserts itself into DNA and prevents DNA unwinding by interfering with the enzyme topoisomerase II.

Imatinib is a tyrosine-kinase inhibitor used in the treatment of multiple cancers, such as Philadelphia chromosome-positive (Ph+) chronic myelogenous leukemia (CML). Imatinib blocks the BCR-Abl enzyme, and stops it from adding phosphate groups. As a result, cells stop growing, and undergo apoptosis. Because the BCR-Abl tyrosine kinase enzyme exists only in cancer cells and not in healthy cells, imatinib works as a form of targeted therapy—only cancer cells are killed through the drug's action.

Interferon alfa enhances the proliferation of human B cells, as well as being able to activate NK cells.

Irinotecan is a chemotherapeutic that prevents DNA from unwinding by inhibition of topoisomerase 1.

Mechlorethamine is the prototype of alkylating agents, a group of anticancer chemotherapeutic drugs. Mechlorethamine works by binding to DNA, crosslinking two strands and preventing cell duplication.

Mercaptopurine is an immunosuppressive medication used to treat acute lymphocytic leukemia. Mercaptopurine interferes with nucleotide synthesis.

Methotrexate belongs to the class of chemotherapy drugs called antimetabolites. Methotrexate exerts its chemotherapeutic effect by being able to counteract and compete with folic acid in cancer cells resulting in folic acid deficiency in the cells and causing their death.

Mitoxantrone is a type II topoisomerase inhibitor that disrupts DNA synthesis and DNA repair in both healthy cells and cancer cells by intercalation between the DNA bases.

Oxaliplatin features a square planar platinum(II) center. In contrast to cisplatin and carboplatin, oxaliplatin features the bidentate ligand 1,2-diaminocyclohexane in place of the two monodentate ammine ligands. It also features a bidentate oxalate group. According to in vivo studies, oxaliplatin fights carcinoma of the colon through non-targeted cytotoxic effects Like other platinum compounds, its cytotoxicity is thought to result from inhibition of DNA synthesis in cells. In particular, oxaliplatin forms both inter- and intra-strand cross links in DNA, which prevent DNA replication and transcription, causing cell death.

Paclitaxel is a chemotherapeutic that targets tubulin. Paclitaxel-treated cells have defects in mitotic spindle assembly, chromosome segregation, and cell division. Unlike other tubulin-targeting drugs such as colchicine that inhibit microtubule assembly, paclitaxel stabilizes the microtubule polymer and protects it from disassembly. Chromosomes are thus unable to achieve a metaphase spindle configuration. This blocks progression of mitosis, and prolonged activation of the mitotic checkpoint triggers apoptosis or reversion to the G-phase of the cell cycle without cell division.

Pemetrexed is a chemotherapy drug in the class of chemotherapy drugs called folate antimetabolites. It works by inhibiting three enzymes used in purine and pyrimidine synthesis—thymidylate synthase (TS), dihydrofolate reductase (DHFR), and glycinamide ribonucleotide formyltransferase (GARFT). By inhibiting the formation of precursor purine and pyrimidine nucleotides, pemetrexed prevents the formation of DNA and RNA, which are required for the growth and survival of both normal cells and cancer cells.

Teniposide is a chemotherapeutic that causes dose-dependent single- and double-stranded breaks in DNA and DNA-protein cross-links. Teniposide has been found to act as an inhibitor of topoisomerase II. The cytotoxic effects of teniposide are related to the relative number of double-stranded DNA breaks produced in cells, which are a reflection of the stabilization of a topoisomerase II-DNA intermediate.

Topotecan is a cancer drug that is a topoisomerase inhibitor. Valrubicin is a chemotherapy drug used to treat bladder cancer. Valrubicin is a semisynthetic analog of the anthracycline doxorubicin, and is administered by infusion directly into the bladder.

The cancer drug may be an anti-apoptotic inhibitors, such as venetoclax. Venetoclax (a BH3-mimetic) is a small molecule that acts as a Bcl-2 inhibitor. Venetoclax blocks the anti-apoptotic B-cell lymphoma-2 (BCL2) protein, leading to programmed cell death in CLL cells.

Vinblastine is a chemotherapeutic that inhibits mitosis. Vinblastine suppresses microtubule dynamics and reduces microtubule polymer mass.

Vincristine is a chemotherapeutic that works partly by binding to the tubulin protein, stopping the cell from separating its chromosomes during the metaphase; the cell then undergoes apoptosis.

Vindesine is an anti-mitotic vinca alkaloid used in chemotherapy.

Vinorelbine is a chemotherapeutic that inhibits mitosis through interaction with tubulin.

In some embodiments, the cancer therapeutic is a monoclonal antibody.

Rituximab (trade names Rituxan, MabThera and Zytux) is a chimeric monoclonal antibody against the protein CD20, which is primarily found on the surface of immune system B cells. Rituximab destroys B cells and is therefore used to treat diseases which are characterized by excessive numbers of B cells, overactive B cells, or dysfunctional B cells. This includes many lymphomas, leukemias, transplant rejection, and autoimmune disorders.

Bevacizumab is a recombinant humanized monoclonal antibody that blocks angiogenesis by inhibiting vascular endothelial growth factor A (VEGF-A). VEGF-A is a chemical signal that stimulates angiogenesis in a variety of diseases, especially in cancer. Bevacizumab was the first clinically available angiogenesis inhibitor in the United States.

Pembrolizumab (formerly MK-3475 and lambrolizumab, trade name Keytruda[1]) is a humanized antibody used in cancer immunotherapy. It targets the programmed cell death 1 (PD-1) receptor.

In certain embodiments, the cancer therapeutic comprises an immune checkpoint inhibitor such as anti-PD-1 or anti-VEGF. A suitable anti-PD-1 may include. Nivolumab (ONO-4538, BMS-936558, or MDX1106) is a human IgG4 anti-PD-1 monoclonal antibody that acts as an immunomodulator by blocking ligand activation of the programmed cell death 1 (PD-1) receptor on activated T cells.

In certain embodiments, the cancer therapeutic comprises a recombinant cytokine such as Interleukin 2 (IL-2), Interleukin 11 (IL-11), or Interleukin 15 (IL-15). IL2 is a lymphokine that induces the proliferation of responsive T cells. In addition, it acts on some B cells, via receptor-specific binding, as a growth factor and antibody production stimulant.

Interleukin 11 (IL-11) is a secreted protein that stimulates megakaryocytopoiesis, resulting in increased production of platelets, as well as activating osteoclasts, inhibiting epithelial cell proliferation and apoptosis, and inhibiting macrophage mediator production.

Interleukin 15 (IL-15) is a cytokine with structural similarity to IL-2. Like IL-2, IL-15 binds to and signals through a complex composed of IL-2/IL-15 receptor beta chain (CD122) and the common gamma chain (gamma-C, CD132). IL-15 is secreted by cells such as mononuclear phagocytes after viral infection. This cytokine induces cell proliferation of natural killer cells; cells of the innate immune system whose principal role is to kill virally infected cells.

v. Introduce to Cell

Methods of the invention include introducing to cells of a patient a treatment that includes: a nuclease or a vector that encodes the nuclease; and a cancer drug. The nuclease is targeted to oncoviral nucleic acid by means of the sequence-specific targeting moiety and it will cleave the viral nucleic acid without interfering with a host genome. Any suitable method can be used to deliver the treatment to the cells. For example, the treatment (or either part of it) may be delivered by injection, orally, or by hydrodynamic delivery. The nuclease or the gene encoding the nuclease may be delivered to systematic circulation or may be delivered or otherwise localized to a specific tissue type. The nuclease or gene encoding the nuclease may be modified or programmed to be active under only certain conditions such as by using a tissue-specific promoter so that the encoded nuclease is preferentially or only transcribed in certain tissue types.

In some embodiments, specific CRISPR/Cas9/gRNA complexes are introduced into a cell. A guide RNA is designed to target at least one category of sequences of the viral genome. In addition to latent infections this invention can also be used to control actively replicating viruses by targeting the viral genome before it is packaged or after it is ejected.

In some embodiments, a cocktail of guide RNAs may be introduced into a cell. The guide RNAs are designed to target numerous categories of sequences of the viral genome. By targeting several areas along the genome, the double strand break at multiple locations fragments the genome, lowering the possibility of repair. Even with repair mechanisms, the large deletions render the virus incapacitated.

In some embodiments, several guide RNAs are added to create a cocktail to target different categories of sequences. For example, two, five, seven or eleven guide RNAs may be present in a CRISPR cocktail targeting three different categories of sequences. However, any number of gRNAs may be introduced into a cocktail to target categories of sequences. In preferred embodiments, the categories of sequences are important for genome structure, host cell transformation, and infection latency, respectively.

In some aspects of the invention, in vitro experiments allow for the determination of the most essential targets within a viral genome. For example, to understand the most essential targets for effective incapacitation of a genome, subsets of guide RNAs are transfected into model cells. Assays can determine which guide RNAs or which cocktail is the most effective at targeting essential categories of sequences.

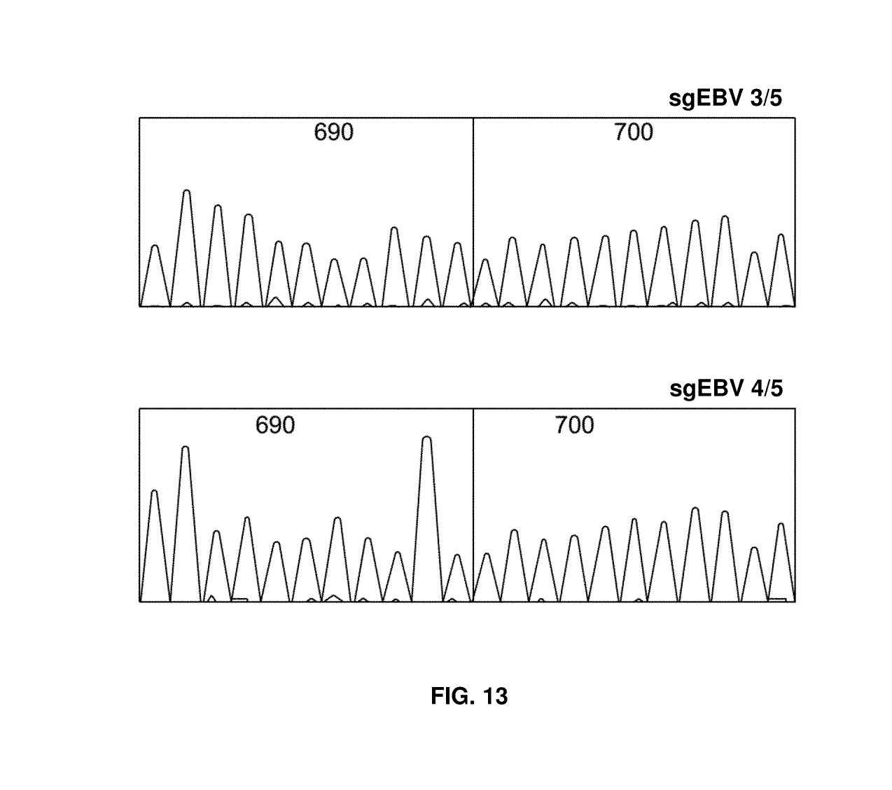

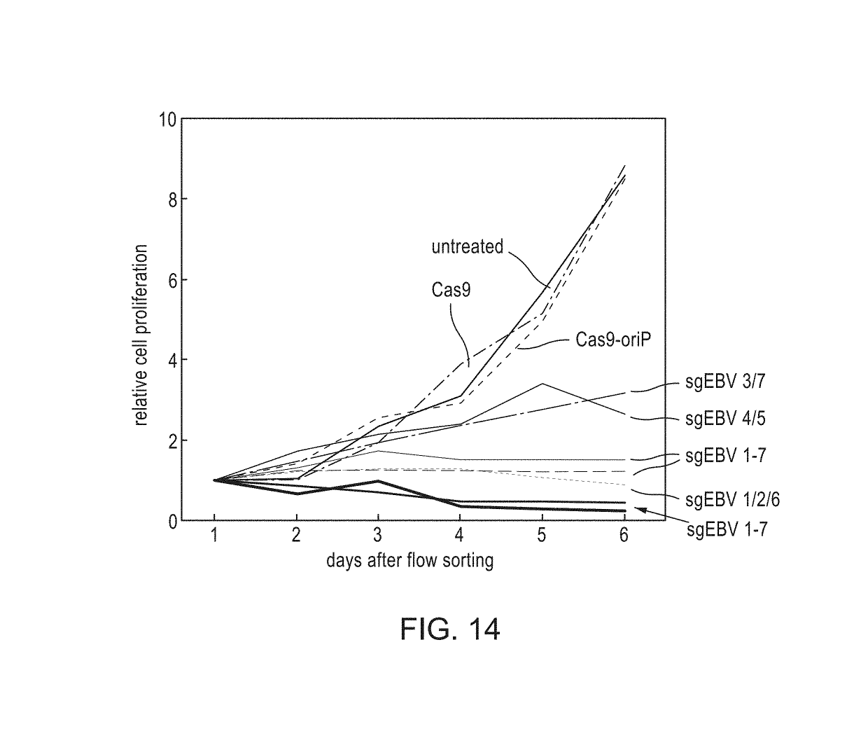

For example, in the case of the EBV genome targeting, seven guide RNAs in the CRISPR cocktail targeted three different categories of sequences which are identified as being important for EBV genome structure, host cell transformation, and infection latency, respectively. To understand the most essential targets for effective EBV treatment, Raji cells were transfected with subsets of guide RNAs. Although sgEBV4/5 reduced the EBV genome by 85%, they could not suppress cell proliferation as effectively as the full cocktail (FIG. 14). Guide RNAs targeting the structural sequences (sgEBV1/2/6) could stop cell proliferation completely, despite not eliminating the full EBV load (26% decrease). Given the high efficiency of genome editing and the proliferation arrest, it was suspect that the residual EBV genome signature in sgEBV1/2/6 was not due to intact genomes but to free-floating DNA that has been digested out of the EBV genome, i.e. as a false positive.

Additionally, recent data suggest that use of ribonucleoprotein (instead of delivery as plasmid DNA) may be preferred for its resulting better DNA cleavage and less off-target cytotox. Recent data suggest that EBV may be effectively targeted using only two EBV guide RNAs, sgEBV2/6. The data suggest that in mixed cell studies with EBV+ cells (Raji) and EBV− cells (DG-75), compositions and methods described herein may exhibit viral specificity of cytotoxicity, preferentially killing infected cells.

Once CRISPR/Cas9/gRNA complexes are constructed, the complexes are introduced into a cell. It should be appreciated that complexes can be introduced into cells in an in vitro model or an in vivo model. In an aspect of the invention, CRISPR/Cas9/gRNA complexes are designed to not leave intact genomes of a virus after transfection and complexes are designed for efficient transfection.

Aspects of the invention allow for CRISPR/Cas9/gRNA to be transfected into cells by various methods, including viral vectors and non-viral vectors. Viral vectors may include retroviruses, lentiviruses, adenoviruses, and adeno-associated viruses. It should be appreciated that any viral vector may be incorporated into the present invention to effectuate delivery of the CRISPR/Cas9/gRNA complex into a cell. Some viral vectors may be more effective than others, depending on the CRISPR/Cas9/gRNA complex designed for digestion or incapacitation. In an aspect of the invention, the vectors contain essential components such as origin of replication, which is necessary for the replication and maintenance of the vector in the host cell.

In an aspect of the invention, viral vectors are used as delivery vectors to deliver the complexes into a cell. Use of viral vectors as delivery vectors are known in the art. See for example U.S. Pub. 2009/0017543 to Wilkes et al., the contents of which are incorporated by reference.

A retrovirus is a single-stranded RNA virus that stores its nucleic acid in the form of an mRNA genome (including the 5′ cap and 3′ PolyA tail) and targets a host cell as an obligate parasite. In some methods in the art, retroviruses have been used to introduce nucleic acids into a cell. Once inside the host cell cytoplasm the virus uses its own reverse transcriptase enzyme to produce DNA from its RNA genome, the reverse of the usual pattern, thus retro (backwards). This new DNA is then incorporated into the host cell genome by an integrase enzyme, at which point the retroviral DNA is referred to as a provirus. For example, the recombinant retroviruses such as the Moloney murine leukemia virus have the ability to integrate into the host genome in a stable fashion. They contain a reverse transcriptase that allows integration into the host genome. Retroviral vectors can either be replication-competent or replication-defective. In some embodiments of the invention, retroviruses are incorporated to effectuate transfection into a cell, however the CRISPR/Cas9/gRNA complexes are designed to target the viral genome.

In some embodiments of the invention, lentiviruses, which are a subclass of retroviruses, are used as viral vectors. Lentiviruses can be adapted as delivery vehicles (vectors) given their ability to integrate into the genome of non-dividing cells, which is the unique feature of lentiviruses as other retroviruses can infect only dividing cells. The viral genome in the form of RNA is reverse-transcribed when the virus enters the cell to produce DNA, which is then inserted into the genome at a random position by the viral integrase enzyme. The vector, now called a provirus, remains in the genome and is passed on to the progeny of the cell when it divides.

As opposed to lentiviruses, adenoviral DNA does not integrate into the genome and is not replicated during cell division. Adenovirus and the related AAV may be used as delivery vectors since they do not integrate into the host's genome. In some aspects of the invention, only the viral genome to be targeted is effected by the CRISPR/Cas9/gRNA complexes, and not the host's cells.

Adeno-associated virus (AAV) is a small virus that infects humans and some other primate species. AAV can infect both dividing and non-dividing cells and may incorporate its genome into that of the host cell. For example, because of its potential use as a gene therapy vector, researchers have created an altered AAV called self-complementary adeno-associated virus (scAAV). Whereas AAV packages a single strand of DNA and requires the process of second-strand synthesis, scAAV packages both strands which anneal together to form double stranded DNA. By skipping second strand synthesis scAAV allows for rapid expression in the cell. Otherwise, scAAV carries many characteristics of its AAV counterpart. Additionally or alternatively, methods and compositions of the invention may use herpesvirus, poxvirus, alphavirus, or vaccinia virus as a means of delivery vectors.

FIG. 7 illustrates gene delivery with an AAV vector. Using known methods, the nucleic acid is packaged in the adenovirus 601. The viral vector 601 fuses with the cell membrane by binding to adhesion molecules and becomes an endosome 607 within the lipid bi-layer. The vesicle opens in the cytoplasm, releasing the vector and the nucleic acid 101, which is transported to and enters the nucleus.

Vectors derived from some AAV serotypes such as AAV9 can cross the blood-brain barrier and transduce cells of the central nervous system (CNS) following a single intravenous injection. In addition to relying on natural diversity, AAV capsids can be decorated by peptides or “shuffled” to generate novel capsids that suit specific needs. For example, a chimeric AAV capsid “shuffled” from five parental natural AAV capsids was recently found to efficiently transduce human liver cells in a humanized mouse model (Lisowski et al., 2014, Nature 506:382). Similar to AdV vector, rAAV vector can transduce both dividing and non-dividing cells, and the recombinant viral genome stays in host nucleus predominantly as episome. Interestingly, single or multiple copies of rAAV vector genome can circularize in a head-to-tail or head-to-head configuration in host nucleus, thus enhancing stability of the episomal rAAV DNA genome and mediating long-term transgene.

An HSV vector may also be used. HSV is a naturally neurotropic virus. After initial infection in skin or mucous membranes, HSV is taken up by sensory nerve terminals, travels along nerves to neuronal cell bodies, and delivers its DNA genome into nuclei for replication. Therefore, HSV vectors are well suited for delivery to neurons.

In certain embodiments of the invention, non-viral vectors may be used to effectuate transfection. Methods of non-viral delivery of nucleic acids include lipofection, nucleofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam and Lipofectin). Cationic and neutral lipids that are suitable for efficient receptor-recognition lipofection of polynucleotides include those described in U.S. Pat. No. 7,166,298 to Jessee or U.S. Pat. No. 6,890,554 to Jesse, the contents of each of which are incorporated by reference. Delivery can be to cells (e.g. in vitro or ex vivo administration) or target tissues (e.g. in vivo administration).

Synthetic vectors are typically based on cationic lipids or polymers which can complex with negatively charged nucleic acids to form particles with a diameter in the order of 100 nm. The complex protects nucleic acid from degradation by nuclease. Moreover, cellular and local delivery strategies have to deal with the need for internalization, release, and distribution in the proper subcellular compartment. Systemic delivery strategies encounter additional hurdles, for example, strong interaction of cationic delivery vehicles with blood components, uptake by the reticuloendothelial system, kidney filtration, toxicity and targeting ability of the carriers to the cells of interest. Modifying the surfaces of the cationic non-virals can minimize their interaction with blood components, reduce reticuloendothelial system uptake, decrease their toxicity and increase their binding affinity with the target cells. Binding of plasma proteins (also termed opsonization) is the primary mechanism for RES to recognize the circulating nanoparticles. For example, macrophages, such as the Kupffer cells in the liver, recognize the opsonized nanoparticles via the scavenger receptor.

In some embodiments of the invention, non-viral vectors are modified to effectuate targeted delivery and transfection. PEGylation (i.e. modifying the surface with polyethyleneglycol) is the predominant method used to reduce the opsonization and aggregation of non-viral vectors and minimize the clearance by reticuloendothelial system, leading to a prolonged circulation lifetime after intravenous administration. PEGylated nanoparticles are therefore often referred as “stealth” nanoparticles. The nanoparticles that are not rapidly cleared from the circulation will have a chance to encounter infected cells.

However, PEG on the surface can decrease the uptake by target cells and reduce the biological activity. Therefore, to attach targeting ligand to the distal end of the PEGylated component is necessary; the ligand is projected beyond the PEG “shield” to allow binding to receptors on the target cell surface.

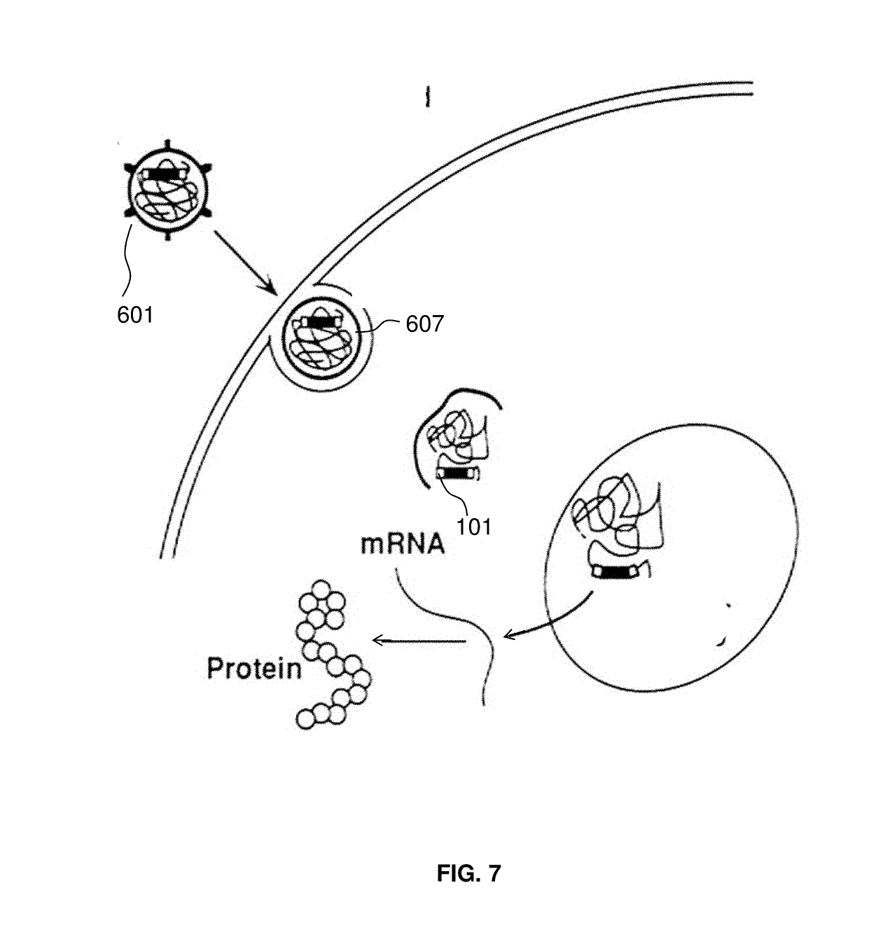

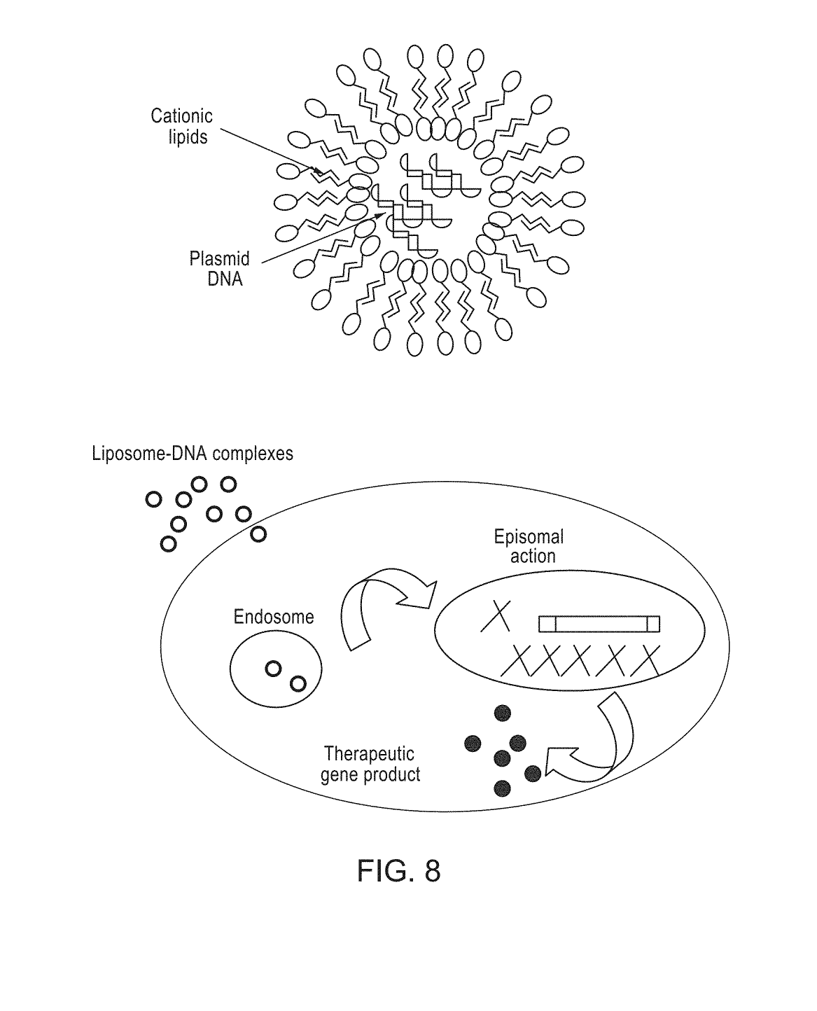

FIG. 8 shows a cationic lipid complex and shows the use of cationic lipids to create a liposome for delivery (although other lipid complexes and compositions are within the scope of the invention) and delivery by liposome. When cationic liposome is used as gene carrier, the application of neutral helper lipid is helpful for the release of nucleic acid, besides promoting hexagonal phase formation to enable endosomal escape. In some embodiments of the invention, neutral or anionic liposomes are developed for systemic delivery of nucleic acids and obtaining therapeutic effect in experimental animal model. Designing and synthesizing novel cationic lipids and polymers, and covalently or non-covalently binding gene with peptides, targeting ligands, polymers, or environmentally sensitive moieties also attract many attentions for resolving the problems encountered by non-viral vectors. The application of inorganic nanoparticles (for example, metallic nanoparticles, iron oxide, calcium phosphate, magnesium phosphate, manganese phosphate, double hydroxides, carbon nanotubes, and quantum dots) in delivery vectors can be prepared and surface-functionalized in many different ways.

In some embodiments, the complexes are conjugated to nano-systems for systemic therapy, such as liposomes, albumin-based particles, PEGylated proteins, biodegradable polymer-drug composites, polymeric micelles, dendrimers, among others. See Davis et al., 2008, Nanotherapeutic particles: an emerging treatment modality for cancer, Nat Rev Drug Discov. 7(9):771-782, incorporated by reference. Long circulating macromolecular carriers such as liposomes, can exploit the enhanced permeability and retention effect for preferential extravasation from tumor vessels. In certain embodiments, the complexes of the invention are conjugated to or encapsulated into a liposome or polymerosome for delivery to a cell. For example, liposomal anthracyclines have achieved highly efficient encapsulation, and include versions with greatly prolonged circulation such as liposomal daunorubicin and pegylated liposomal doxorubicin. See Krishna et al., Carboxymethylcellulose-sodium based transdermal drug delivery system for propranolol, J Pharm Pharmacol. 1996 April; 48(4):367-70.

Liposomal delivery systems provide stable formulation, provide improved pharmacokinetics, and a degree of ‘passive’ or ‘physiological’ targeting to tissues. Encapsulation of hydrophilic and hydrophobic materials, such as potential cancer drugs, are known. See for example U.S. Pat. No. 5,466,468; U.S. Pat. No. 5,580,571; U.S. Pat. No. 5,626,869, the contents of each of which are incorporated by reference.

Liposomes and polymerosomes can contain a plurality of solutions and compounds. In certain embodiments, the complexes of the invention are coupled to or encapsulated in polymersomes. As a class of artificial vesicles, polymersomes are tiny hollow spheres that enclose a solution, made using amphiphilic synthetic block copolymers to form the vesicle membrane. Common polymersomes contain an aqueous solution in their core and are useful for encapsulating and protecting sensitive molecules, such as drugs, enzymes, other proteins and peptides, and DNA and RNA fragments. The polymersome membrane provides a physical barrier that isolates the encapsulated material from external materials, such as those found in biological systems. Polymerosomes can be generated from double emulsions by known techniques, see Lorenceau et al., 2005, Generation of Polymerosomes from Double-Emulsions, Langmuir 21(20):9183-6, incorporated by reference.

Aspects of the invention provide for numerous uses of delivery vectors. Selection of the delivery vector is based upon the cell or tissue targeted and the specific makeup of the CRISPR/Cas9/gRNA. For example, in the EBV example discussed above, since lymphocytes are known for being resistant to lipofection, nucleofection (a combination of electrical parameters generated by a device called Nucleofector, with cell-type specific reagents to transfer a substrate directly into the cell nucleus and the cytoplasm) was necessitated for DNA delivery into the Raji cells. The Lonza pmax promoter drives Cas9 expression as it offered strong expression within Raji cells. 24 hours after nucleofection, obvious EGFP signals were observed from a small proportion of cells through fluorescent microscopy. The EGFP-positive cell population decreased dramatically, however, <10% transfection efficiency 48 hours after nucleofection was measured.

Aspects of the invention use the CRISPR/Cas9/gRNA complexes and targeted delivery. Common known pathways include transdermal, transmucal, nasal, ocular and pulmonary routes. Drug delivery systems may include liposomes, proliposomes, microspheres, gels, prodrugs, cyclodextrins, etc. Aspects of the invention utilize nanoparticles composed of biodegradable polymers to be transferred into an aerosol for targeting of specific sites or cell populations in the lung, providing for the release of the drug in a predetermined manner and degradation within an acceptable period of time. Controlled-release technology, such as transdermal and transmucosal controlled-release delivery systems, nasal and buccal aerosol sprays, drug-impregnated lozenges, encapsulated cells, oral soft gels, iontophoretic devices to administer drugs through skin, and a variety of programmable, implanted drug-delivery devices are used in conjunction with the complexes of the invention of accomplishing targeted and controlled delivery.

vi. Cut Nucleic Acid

Once inside the cell, the nuclease targets oncoviral nucleic acid sequences. In some embodiments, methods and compositions of the invention use a nuclease such as Cas9 to target latent oncoviral genomes, thereby reducing the chances of proliferation.

Upon introduction of Cas9 nuclease into target cells, the nuclease forms a complex with the gRNA (e.g., crRNA+tracrRNA or sgRNA). The complex cuts the viral nucleic acid in a targeted fashion to incapacitate the viral genome. The Cas9 endonuclease causes a double strand break in the viral genome. By targeted several locations along the viral genome and causing not a single strand break, but a double strand break, the genome is effectively cut a several locations along the genome. In a preferred embodiment, the double strand breaks are designed so that small deletions are caused, or small fragments are removed from the genome so that even if natural repair mechanisms join the genome together, the genome is render incapacitated.

The nuclease, or a gene encoding the nuclease, may be delivered to cells by transfection. For example, the cells may be transfected with DNA that encodes Cas9 and gRNA (on a single piece or separate pieces). The gRNAs are designed to localize the Cas9 endonuclease at one or several locations along the viral genome. The Cas9 endonuclease causes double strand breaks in the genome, causing small fragments to be deleted from the viral genome. Even with repair mechanisms, the deletions render the viral genome incapacitated.

vii. Host Genome

It will be appreciated that method and compositions of the invention can be used to target oncoviral nucleic acid without interfering with host genetic material. Methods and compositions of the invention employ a targeting moiety such as a guide RNA that has a sequence that hybridizes to a target within the viral sequence. Methods and compositions of the invention may further use a targeted nuclease such as the cas9 enzyme, or a vector encoding such a nuclease, which uses the gRNA to bind exclusively to the viral genome and make double stranded cuts, thereby removing the viral sequence from the host.

Where the targeting moiety includes a guide RNA, the sequence for the gRNA, or the guide sequence, can be determined by examination of the viral sequence to find regions of about 20 nucleotides that are adjacent to a protospacer adjacent motif (PAM) and that do not also appear in the host genome adjacent to the protospacer motif.

Preferably a guide sequence that satisfies certain similarity criteria (e.g., at least 60% identical with identity biased toward regions closer to the PAM) so that a gRNA/cas9 complex made according to the guide sequence will bind to and digest specified features or targets in the viral sequence without interfering with the host genome. Preferably, the guide RNA corresponds to a nucleotide string next to a protospacer adjacent motif (PAM) (e.g., NGG, where N is any nucleotide) in the viral sequence. Preferably, the host genome lacks any region that (1) matches the nucleotide string according to a predetermined similarity criteria and (2) is also adjacent to the PAM. The predetermined similarity criteria may include, for example, a requirement of at least 12 matching nucleotides within 20 nucleotides 5′ to the PAM and may also include a requirement of at least 7 matching nucleotides within 10 nucleotides 5′ to the PAM. An annotated viral genome (e.g., from GenBank) may be used to identify features of the viral sequence and finding the nucleotide string next to a protospacer adjacent motif (PAM) in the viral sequence within a selected feature (e.g., a viral replication origin, a terminal repeat, a replication factor binding site, a promoter, a coding sequence, or a repetitive region) of the viral sequence. The viral sequence and the annotations may be obtained from a genome database.

Where multiple candidate gRNA targets are found in the viral genome, selection of the sequence to be the template for the guide RNA may favor the candidate target closest to, or at the 5′ most end of, a targeted feature as the guide sequence. The selection may preferentially favor sequences with neutral (e.g., 40% to 60%) GC content. Additional background regarding the RNA-directed targeting by endonuclease is discussed in U.S. Pub. 2015/0050699; U.S. Pub. 20140356958; U.S. Pub. 2014/0349400; U.S. Pub. 2014/0342457; U.S. Pub. 2014/0295556; and U.S. Pub. 2014/0273037, the contents of each of which are incorporated by reference for all purposes. Due to the existence of human genomes background in the infected cells, a set of steps are provided to ensure high efficiency against the viral genome and low off-target effect on the human genome. Those steps may include (1) target selection within viral genome, (2) avoiding PAM+target sequence in host genome, (3) methodologically selecting viral target that is conserved across strains, (4) selecting target with appropriate GC content, (5) control of nuclease expression in cells, (6) vector design, (7) validation assay, others and various combinations thereof. A targeting moiety (such as a guide RNA) preferably binds to targets within certain categories such as (i) latency related targets, (ii) infection and symptom related targets, and (iii) structure related targets.

A first category of targets for gRNA includes latency-related targets. The viral genome requires certain features in order to maintain the latency. These features include, but not limited to, master transcription regulators, latency-specific promoters, signaling proteins communicating with the host cells, etc. If the host cells are dividing during latency, the viral genome requires a replication system to maintain genome copy level. Viral replication origin, terminal repeats, and replication factors binding to the replication origin are great targets. Once the functions of these features are disrupted, the viruses may reactivate, which can be treated by conventional antiviral therapies.

A second category of targets for gRNA includes infection-related and symptom-related targets. Virus produces various molecules to facilitate infection. Once gained entrance to the host cells, the virus may start lytic cycle, which can cause cell death and tissue damage (HBV). In certain cases, such as HPV-16 or HPV-18, cell products (E6 and E7 proteins) can transform the host cells and cause cancers. E6 from HPV-18 is reported as an oncogene capable of transforming cells and thus provides a target according to certain embodiments. Disrupting the key genome sequences (promoters, coding sequences, etc) producing these molecules can prevent further infection, and/or relieve symptoms, if not curing the disease.

A third category of targets for gRNA includes structure-related targets. Viral genome may contain repetitive regions to support genome integration, replication, or other functions. Targeting repetitive regions can break the viral genome into multiple pieces, which physically destroys the genome.

Where the nuclease is a cas protein, the targeting moiety is a guide RNA. Each cas protein requires a specific PAM next to the targeted sequence (not in the guide RNA). This is the same as for human genome editing. The current understanding the guide RNA/nuclease complex binds to PAM first, then searches for homology between guide RNA and target genome. Sternberg et al., 2014, DNA interrogation by the CRISPR RNA-guided endonuclease Cas9, Nature 507(7490):62-67. Once recognized, the DNA is digested 3-nt upstream of PAM. These results suggest that off-target digestion requires PAM in the host DNA, as well as high affinity between guide RNA and host genome right before PAM.

It may be preferable to use a targeting moiety that targets portions of the viral genome that are highly conserved. Viral genomes are much more variable than human genomes. In order to target different strains, the guide RNA will preferably target conserved regions. As PAM is important to initial sequence recognition, it is also essential to have PAM in the conserved region.

In a preferred embodiment, methods of the invention are used to deliver a nucleic acid to cells. The nucleic acid delivered to the cells may include a gRNA having the determined guide sequence or the nucleic acid may include a vector, such as a plasmid, that encodes an enzyme that will act against the target genetic material. Expression of that enzyme allows it to degrade or otherwise interfere with the target genetic material. The enzyme may be a nuclease such as the Cas9 endonuclease and the nucleic acid may also encode one or more gRNA having the determined guide sequence.

The gRNA targets the nuclease to the target genetic material. Where the target genetic material includes the genome of a virus, gRNAs complementary to parts of that genome can guide the degredation of that genome by the nuclease, thereby preventing any further replication or even removing any intact viral genome from the cells entirely. By these means, latent viral infections can be targeted for eradication.

The host cells may grow at different rate, based on the specific cell type. High nuclease expression is necessary for fast replicating cells, whereas low expression help avoiding off-target cutting in non-infected cells. Control of nuclease expression can be achieved through several aspects. If the nuclease is expressed from a vector, having the viral replication origin in the vector can increase the vector copy number dramatically, only in the infected cells. Each promoter has different activities in different tissues. Gene transcription can be tuned by choosing different promoters. Transcript and protein stability can also be tuned by incorporating stabilizing or destabilizing (ubiquitin targeting sequence, etc) motif into the sequence.

Specific promoters may be used for the gRNA sequence, the nuclease (e.g., cas9), other elements, or combinations thereof. For example, in some embodiments, the gRNA is driven by a U6 promoter. A vector may be designed that includes a promoter for protein expression (e.g., using a promoter as described in the vector sold under the trademark PMAXCLONING by Lonza Group Ltd (Basel, Switzerland). A vector may be a plasmid (e.g., created by synthesis instrument 255 and recombinant DNA lab equipment). In certain embodiments, the plasmid includes a U6 promoter driven gRNA or chimeric guide RNA (sgRNA) and a ubiquitous promoter-driven cas9. Optionally, the vector may include a marker such as EGFP fused after the cas9 protein to allow for later selection of cas9+ cells. It is recognized that cas9 can use a gRNA (similar to the CRISPR RNA (crRNA) of the original bacterial system) with a complementary trans-activating crRNA (tracrRNA) to target viral sequences complementary to the gRNA. It has also been shown that cas9 can be programmed with a single RNA molecule, a chimera of the gRNA and tracrRNA. The single guide RNA (sgRNA) can be encoded in a plasmid and transcription of the sgRNA can provide the programming of cas9 and the function of the tracrRNA. See Jinek, 2012, A programmable dual-RNA-guided DNA endonuclease in adaptive bacterial immunity, Science 337:816-821 and especially FIG. 5A therein for background.

Using the above principles, methods and compositions of the invention may be used to target viral nucleic acid in an infected host without adversely influencing the host genome.

For additional background see Hsu, 2013, DNA targeting specificity of RNA-guided Cas9 nucleases, Nature Biotechnology 31(9):827-832; and Jinek, 2012, A programmable dual-RNA-guided DNA endonuclease in adaptive bacterial immunity, Science 337:816-821, the contents of each of which are incorporated by reference. Since the targeted locations are selected to be within certain categories such as (i) latency related targets, (ii) infection and symptom related targets, or (iii) structure related targets, cleavage of those sequences inactivates the virus and removes it from the host. Since the targeting RNA (the gRNA or sgRNA) is designed to satisfy according to similarity criteria that matches the target in the viral genetic sequence without any off-target matching the host genome, the latent viral genetic material is removed from the host without any interference with the host genome.

viii. Composition

In some embodiments, the invention provides a composition for topical application (e.g., in vivo, directly to skin of a person). The composition may be applied superficially (e.g., topically). The composition provides a nuclease or gene therefore and includes a pharmaceutically acceptable diluent, adjuvant, or carrier. Preferably, a carrier used in accordance with the subject invention is approved for animal or human use by a competent governmental agency, such as the US Food and Drug Administration (FDA) or the like. Examples include, but are not limited to, phosphate buffered saline, physiological saline, water, and emulsions, such as oil/water emulsions. The carrier can be a solvent or dispersing medium containing, for example, ethanol, polyol (for example, glycerol, propylene glycol, liquid polyethylene glycol, and the like), suitable mixtures thereof, and vegetable oils. These formulations contain from about 0.01% to about 100%, preferably from about 0.01% to about 90% of the MFB extract, the balance (from about 0% to about 99.99%, preferably from about 10% to about 99.99% of an acceptable carrier or other excipients. A more preferred formulation contains up to about 10% MFB extract and about 90% or more of the carrier or excipient, whereas a typical and most preferred composition contains about 5% MFB extract and about 95% of the carrier or other excipients. Formulations are described in a number of sources that are well known and readily available to those skilled in the art.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

### EXAMPLES

### Example 1

### Targeting EBV

Burkitt's lymphoma cell lines Raji, Namalwa, and DG-75 were obtained from ATCC and cultured in RPMI 1640 supplemented with 10% FBS and PSA, following ATCC recommendation. Human primary lung fibroblast IMR-90 was obtained from Coriell and cultured in Advanced DMEM/F-12 supplemented with 10% FBS and PSA.

Plasmids consisting of a U6 promoter driven chimeric guide RNA (sgRNA) and a ubiquitous promoter driven Cas9 were obtained from addgene, as described by Cong L et al. (2013) Multiplex Genome Engineering Using CRISPR/Cas Systems. Science 339:819-823. An EGFP marker fused after the Cas9 protein allowed selection of Cas9-positive cells (FIG. 4). We adapted a modified chimeric guide RNA design for more efficient Pol-III transcription and more stable stem-loop structure (Chen B et al. (2013) Dynamic Imaging of Genomic Loci in Living Human Cells by an Optimized CRISPR/Cas System. Cell 155:1479-1491).

We obtained pX458 from Addgene, Inc. A modified CMV promoter with a synthetic intron (pmax) was PCR amplified from Lonza control plasmid pmax-GFP. A modified guide RNA sgRNA(F+E) was ordered from IDT. EBV replication origin oriP was PCR amplified from B95-8 transformed lymphoblastoid cell line GM12891. We used standard cloning protocols to clone pmax, sgRNA(F+E) and oriP to pX458, to replace the original CAG promoter, sgRNA and f1 origin. We designed EBV sgRNA based on the B95-8 reference, and ordered DNA oligos from IDT. The original sgRNA place holder in pX458 serves as the negative control.

Lymphocytes are known for being resistant to lipofection, and therefore we used nucleofection for DNA delivery into Raji cells. We chose the Lonza pmax promoter to drive Cas9 expression as it offered strong expression within Raji cells. We used the Lonza Nucleofector II for DNA delivery. 5 million Raji or DG-75 cells were transfected with 5 ug plasmids in each 100-ul reaction. Cell line Kit V and program M-013 were used following Lonza recommendation. For IMR-90, 1 million cells were transfected with 5 ug plasmids in 100 ul Solution V, with program T-030 or X-005. 24 hours after nucleofection, we observed obvious EGFP signals from a small proportion of cells through fluorescent microscopy. The EGFP-positive cell population decreased dramatically after that, however, and we measured <10% transfection efficiency 48 hours after nucleofection. We attributed this transfection efficiency decrease to the plasmid dilution with cell division. To actively maintain the plasmid level within the host cells, we redesigned the CRISPR plasmid to include the EBV origin of replication sequence, oriP. With active plasmid replication inside the cells, the transfection efficiency rose to >60%.

To design guide RNA targeting the EBV genome, we relied on the EBV reference genome from strain B95-8. We targeted six regions with seven guide RNA designs for different genome editing purposes. The guide RNAs are listed in Table S1 in Wang and Quake, 2014, RNA-guided endonuclease provides a therapeutic strategy to cure latent herpesviridae infection, PNAS 111(36):13157-13162 and in the Supporting Information to that article published online at the PNAS website, and the contents of both of those documents are incorporated by reference for all purposes.

EBNA1 is crucial for many EBV functions including gene regulation and latent genome replication. We targeted guide RNA sgEBV4 and sgEBV5 to both ends of the EBNA1 coding region in order to excise this whole region of the genome. Guide RNAs sgEBV1, 2 and 6 fall in repeat regions, so that the success rate of at least one CRISPR cut is multiplied. These “structural” targets enable systematic digestion of the EBV genome into smaller pieces. EBNA3C and LMP1 are essential for host cell transformation, and we designed guide RNAs sgEBV3 and sgEBV7 to target the 5′ exons of these two proteins respectively.

### EBV Genome Editing.

The double-strand DNA breaks generated by CRISPR are repaired with small deletions. These deletions will disrupt the protein coding and hence create knockout effects. SURVEYOR assays confirmed efficient editing of individual sites. Beyond the independent small deletions induced by each guide RNA, large deletions between targeting sites can systematically destroy the EBV genome.

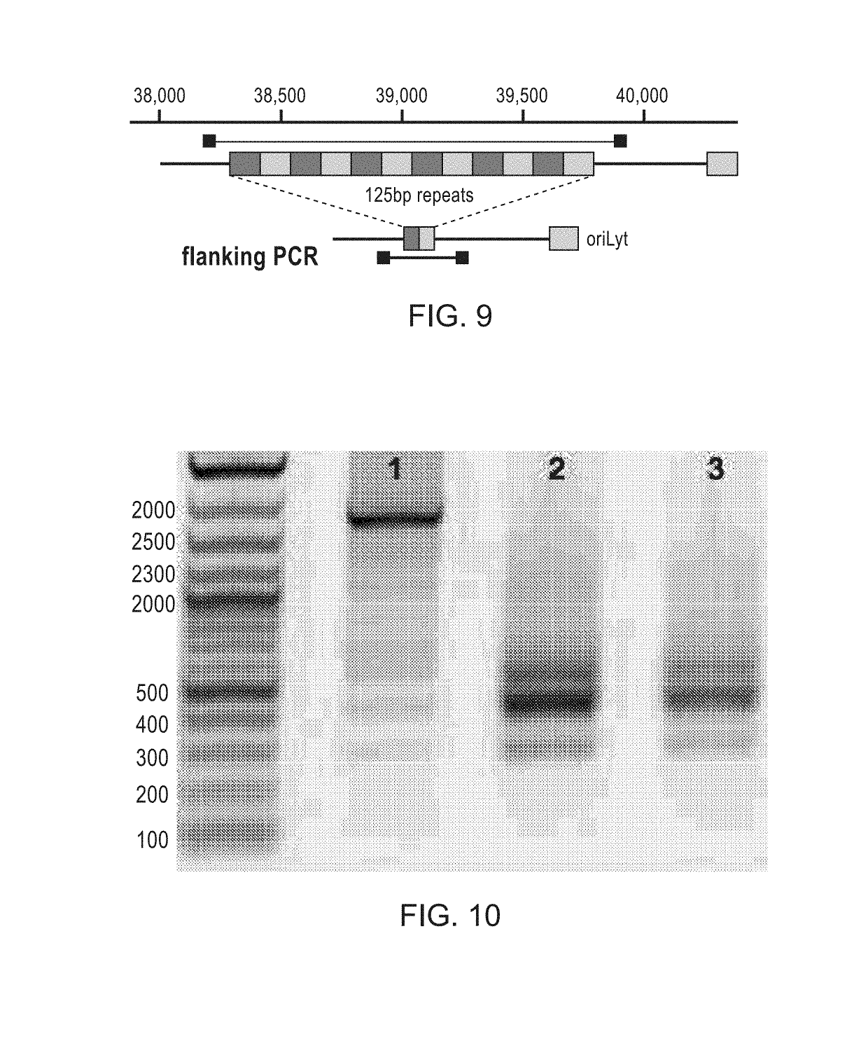

FIG. 9 shows genomic context around guide RNA sgEBV2 and PCR primer locations.

FIG. 10 shows a large deletion induced by sgEBV2, where lane 1-3 are before, 5 days after, and 7 days after sgEBV2 treatment, respectively. Guide RNA sgEBV2 targets a region with twelve 125-bp repeat units (FIG. 9). PCR amplicon of the whole repeat region gave a ˜1.8-kb band (FIG. 10). After 5 or 7 days of sgEBV2 transfection, we obtained ˜0.4-kb bands from the same PCR amplification (FIG. 10). The ˜1.4-kb deletion is the expected product of repair ligation between cuts in the first and the last repeat unit (FIG. 9).

DNA sequences flanking sgRNA targets were PCR amplified with Phusion DNA polymerase. SURVEYOR assays were performed following manufacturer's instruction. DNA amplicons with large deletions were TOPO cloned and single colonies were used for Sanger sequencing. EBV load was measured with Taqman digital PCR on Fluidigm BioMark. A Taqman assay targeting a conserved human locus was used for human DNA normalization. 1 ng of single-cell whole-genome amplification products from Fluidigm C1 were used for EBV quantitative PCR.

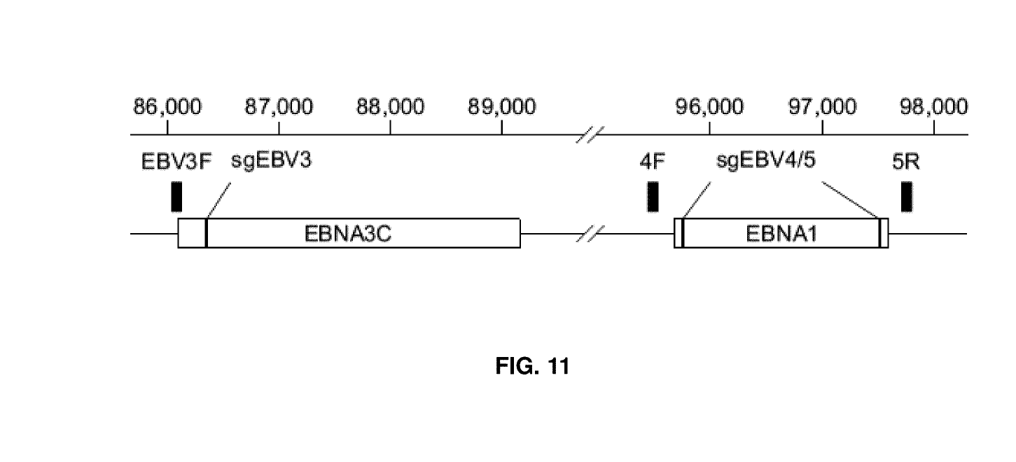

We further demonstrated that it is possible to delete regions between unique targets. FIG. 11 shows the region targeted by sgEBV4/5 (e.g., between the forward (4F) and reverse (5R) primer binding sites). Six days after sgEBV4-5 transfection, PCR amplification of the whole flanking region (with primers EBV4F and 5R) returned a shorter amplicon, together with a much fainter band of the expected 2 kb.

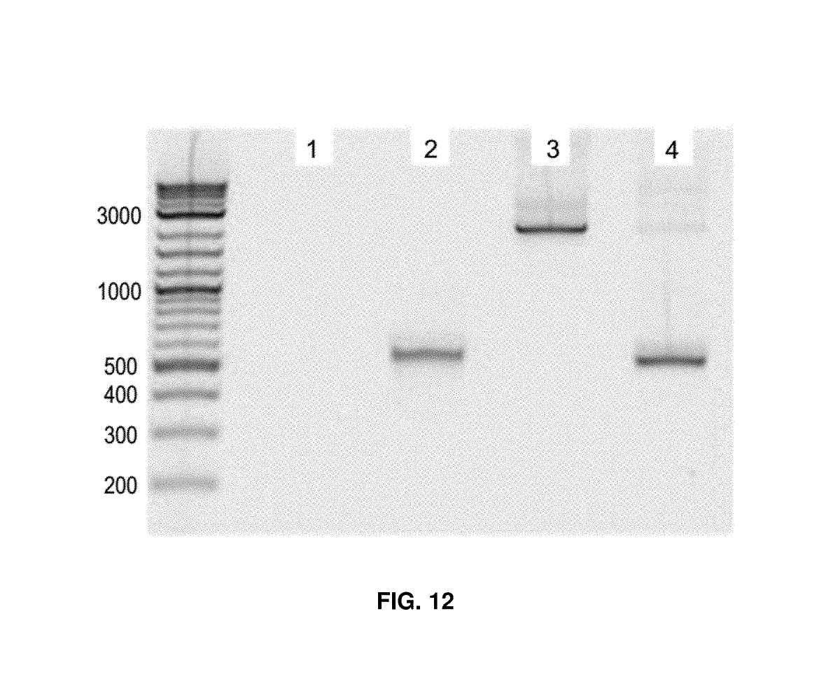

FIG. 12 in lane 4 shows the faint band of the expected 2 kb. Sanger sequencing of amplicon clones confirmed the direct connection of the two expected cutting sites. A similar experiment with sgEBV3-5 also returned an even larger deletion, from EBNA3C to EBNA1.

Additional information such as primer design is shown in Wang and Quake, 2014, RNA-guided endonuclease provides a therapeutic strategy to cure latent herpesviridae infection, PNAS 111(36):13157-13162 and in the Supporting Information to that article published online at the PNAS website, and the contents of both of those documents are incorporated by reference for all purposes.

### Cell Proliferation Arrest With EBV Genome Destruction.

Two days after CRISPR transfection, we flow sorted EGFP-positive cells for further culture and counted the live cells daily. FIG. 11 gives genome context around guide RNA sgEBV3/4/5 and PCR primer locations.

FIG. 12 shows large deletions induced by sgEBV3/5 and sgEBV4/5, where lane 1 and 2 are 3F/5R PCR amplicons before and 8 days after sgEBV3/5 treatment; and lane 3 and 4 are 4F/5R PCR amplicons before and 8 days after sgEBV4/5 treatment.

FIG. 13 shows that Sanger sequencing confirmed: genome cleavage and repair ligation 8 days after sgEBV3/5 treatment (top) and genome cleavage and repair ligation 8 days after sgEBV4/5 treatment (bottom).

FIG. 14 shows several cell proliferation curves after different CRISPR treatments.

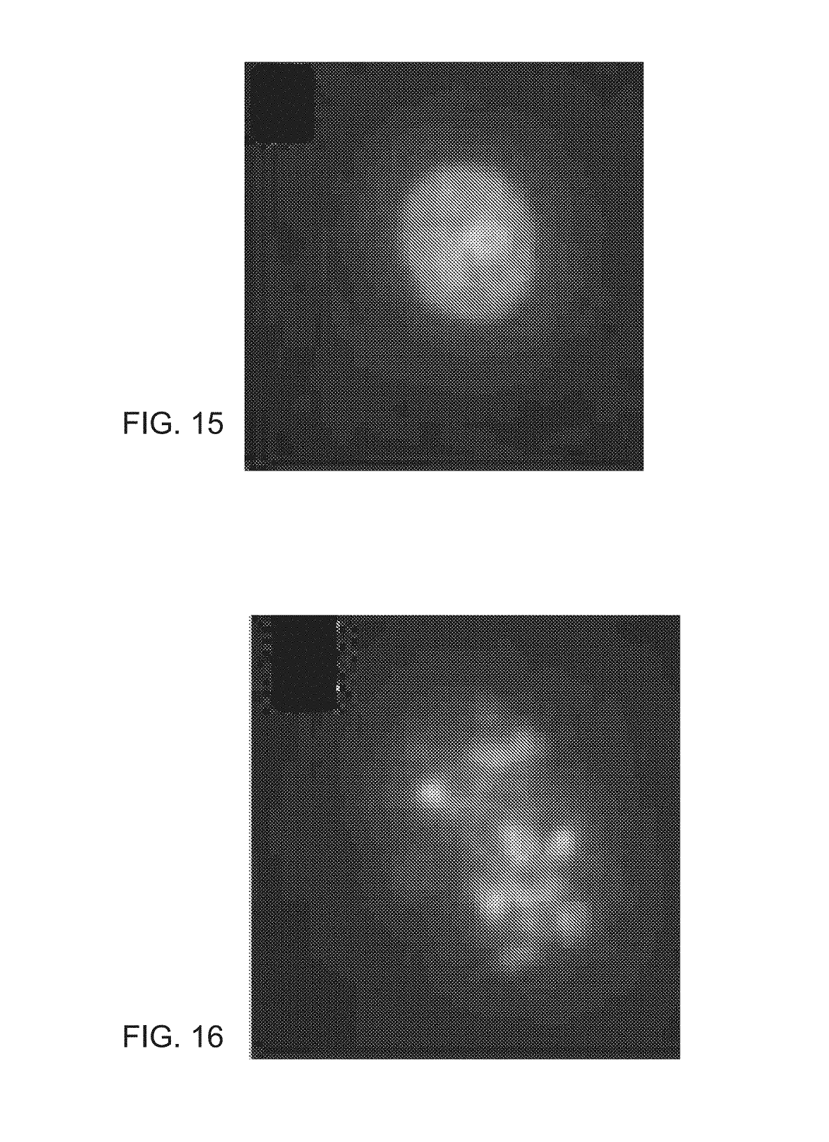

FIG. 15 shows nuclear morphology before sgEBV1-7 treatment.

FIG. 16 shows nuclear morphology after sgEBV1-7 treatment.

As expected, cells treated with Cas9 plasmids which lacked oriP or sgEBV lost EGFP expression within a few days and proliferated with a rate similar rate to the untreated control group (FIG. 14). Plasmids with Cas9-oriP and a scrambled guide RNA maintained EGFP expression after 8 days, but did not reduce the cell proliferation rate. Treatment with the mixed cocktail sgEBV1-7 resulted in no measurable cell proliferation and the total cell count either remained constant or decreased (FIG. 14). Flow cytometry scattering signals clearly revealed alterations in the cell morphology after sgEBV1-7 treatment, as the majority of the cells shrank in size with increasing granulation. Cells in population P3 also demonstrated compromised membrane permeability by DAPI staining.

To rule out the possibility of CRISPR cytotoxicity, especially with multiple guide RNAs, we performed the same treatment on two other samples: the EBV-negative Burkitt's lymphoma cell line DG-75 and primary human lung fibroblast IMR90.

Eight and nine days after transfection the cell proliferation rates did not change from the untreated control groups, suggesting neglectable cytotoxicity.

Previous studies have attributed the EBV tumorigenic ability to its interruption of host cell apoptosis (Ruf I K et al. (1999) Epstein-Barr Virus Regulates c-MYC, Apoptosis, and Tumorigenicity in Burkitt Lymphoma. Molecular and Cellular Biology 19:1651-1660). Suppressing EBV activities may therefore restore the apoptosis process, which could explain the cell death observed in our experiment. Annexin V staining revealed a distinct subpopulation of cells with intact cell membrane but exposed phosphatidylserine, suggesting cell death through apoptosis. Bright field microscopy showed obvious apoptotic cell morphology and fluorescent staining demonstrated drastic DNA fragmentation (FIGS. 15-16). Altogether this evidence suggests restoration of the normal host cell apoptosis pathway after EBV genome destruction.

### Complete Clearance of EBV in a Subpopulation.

To study the potential connection between cell proliferation arrest and EBV genome editing, we quantified the EBV load in different samples with digital PCR targeting EBNA1. Another Taqman assay targeting a conserved human somatic locus served as the internal control for human DNA normalization.

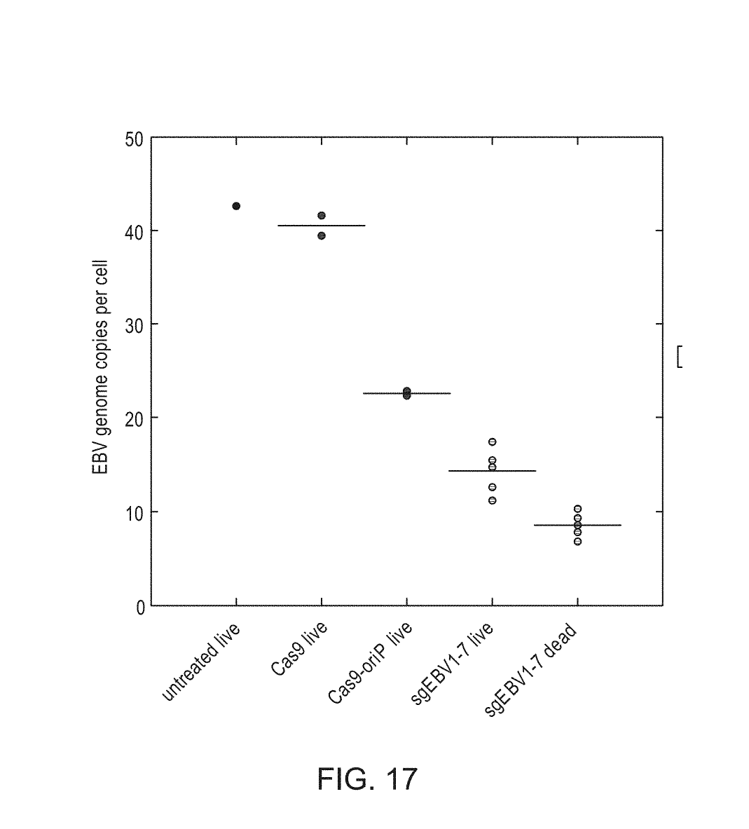

FIG. 17 shows EBV load after different CRISPR treatments by digital PCR, where Cas9 and Cas9-oriP had two replicates, and sgEBV1-7 had 5 replicates.

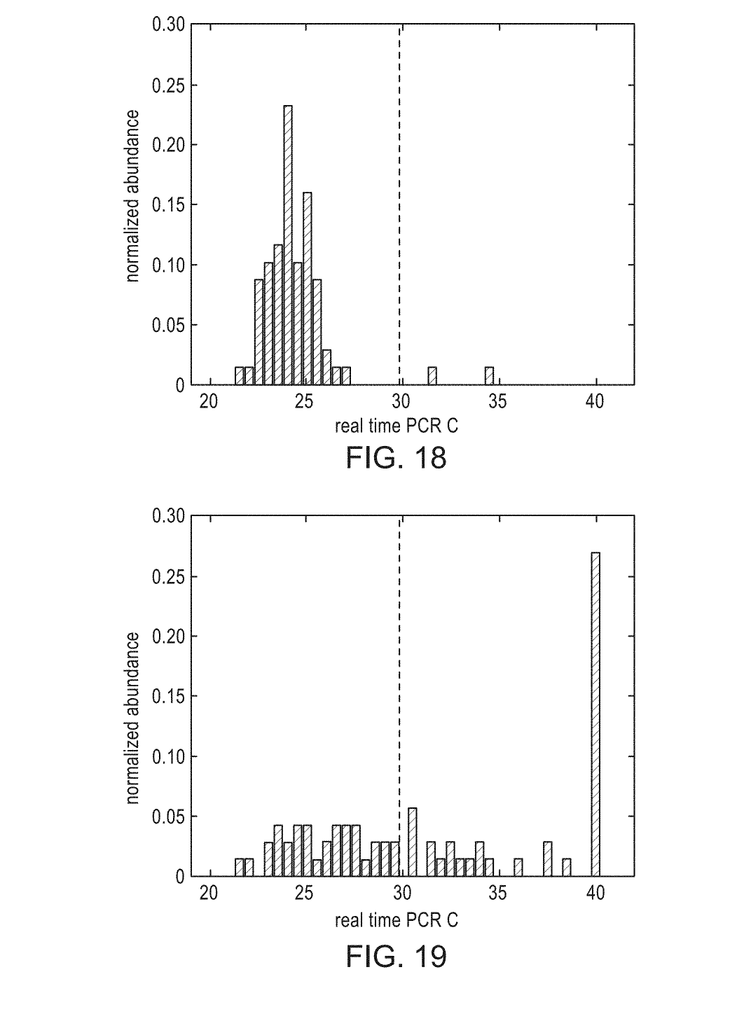

FIG. 18 gives a histogram of EBV quantitative PCR Ct values from single cells before treatment.

FIG. 19 gives a histogram of EBV quantitative PCR Ct values from single live cells 7 days after sgEBV1-7 treatment, where the dash lines in FIGS. 30 & 31 represent Ct values of one EBV genome per cell.

On average, each untreated Raji cell has 42 copies of EBV genome. Cells treated with a Cas9 plasmid that lacked oriP or sgEBV did not have an obvious difference in EBV load difference from the untreated control. Cells treated with a Cas9-plasmid with oriP but no sgEBV had an EBV load that was reduced by ˜50%. In conjunction with the prior observation that cells from this experiment did not show any difference in proliferation rate, we interpret this as likely due to competition for EBNA1 binding during plasmid replication. The addition of the guide RNA cocktail sgEBV1-7 to the transfection dramatically reduced the EBV load. Both the live and dead cells have >60% EBV decrease comparing to the untreated control.

Although we provided seven guide RNAs at the same molar ratio, the plasmid transfection and replication process is likely quite stochastic. Some cells will inevitably receive different subsets or mixtures of the guide RNA cocktail, which might affect the treatment efficiency. To control for such effects, we measured EBV load at the single cell level by employing single-cell whole-genome amplification with an automated microfluidic system. We loaded freshly cultured Raji cells onto the microfluidic chip and captured 81 single cells. For the sgEBV1-7 treated cells, we flow sorted the live cells eight days after transfection and captured 91 single cells. Following manufacturer's instruction, we obtained ˜150 ng amplified DNA from each single cell reaction chamber. For quality control purposes we performed 4-loci human somatic DNA quantitative PCR on each single cell amplification product (Wang et al., 2012, Genome-wide single-cell analysis of recombination activity and de novo mutation rates in human sperm, Cell 150:402-412) and required positive amplification from at least one locus. 69 untreated single-cell products passed the quality control and displayed a log-normal distribution of EBV load with almost every cell displaying significant amounts of EBV genomic DNA. We calibrated the quantitative PCR assay with a subclone of Namalwa Burkitt's lymphoma cells, which contain a single integrated EBV genome. The single-copy EBV measurements gave a Ct of 29.8, which enabled us to determine that the mean Ct of the 69 Raji single cell samples corresponded to 42 EBV copies per cells, in concordance with the bulk digital PCR measurement. For the sgEBV1-7 treated sample, 71 single-cell products passed the quality control and the EBV load distribution was dramatically wider. While 22 cells had the same EBV load as the untreated cells, 19 cells had no detectable EBV and the remaining 30 cells displayed dramatic EBV load decrease from the untreated sample.

Essential Targets For EBV Treatment. The seven guide RNAs in our CRISPR cocktail target three different categories of sequences which are important for EBV genome structure, host cell transformation, and infection latency, respectively. To understand the most essential targets for effective EBV treatment, we transfected Raji cells with subsets of guide RNAs. Although sgEBV4/5 reduced the EBV genome by 85%, they could not suppress cell proliferation as effectively as the full cocktail (FIG. 14). Guide RNAs targeting the structural sequences (sgEBV1/2/6) could stop cell proliferation completely, despite not eliminating the full EBV load (26% decrease). Given the high efficiency of genome editing and the proliferation arrest, we suspect that the residual EBV genome signature in sgEBV1/2/6 was not due to intact genomes but to free-floating DNA that has been digested out of the EBV genome, i.e. as a false positive. We conclude that systematic destruction of EBV genome structure appears to be more effective than targeting specific key proteins for EBV treatment.

### Example 2

### HPV Genome and Targets

The HPV genome is a double-stranded, circular DNA genome approximately 8 kb in size that can be divided, in general, into three major regions (early, late, and a long control region (LCR), which regions are separated by two polyadenylation sites. The early region is over 50% of the HPV genome from its 5′ half and encodes six common open reading frames (E1, E2, E4, ES, E6 and E7) that translate proteins. The late region is downstream of the early region and encodes L1 and L2 ORFs for translation of a major (L1) and a minor (L2) capsid protein. A targeting sequence such as a gRNA may be targeted to a capsid protein to interrupt viral function. The ˜850 bp LCR region has no protein-coding function, but bears the origin of replication as well as transcription factor binding sites for transcription regulation from viral early as well as late promoters. See Bernard, 2007, Gene expression of genital human papillomaviruses and considerations on potential antiviral approaches. Antivir. Ther. 7:219-237 incorporated by reference. The HPV-16 genome contains two major promoters. The P97 promoter lies upstream of the E6 ORF and is responsible for almost all early gene expression. The P670 promoter lies within the E7 ORF region and is responsible for late gene expression. The HPV-16 P97 promoter, equivalent to P99 in HPV-31 and P105 in HPV-18, is very potent and tightly controlled, primarily by upstream cis-elements in the LCR that interact with cellular transcription factors and the viral transactivator/repressor E2 and regulate the transcription of P97 from undifferentiated basal cells to highly differentiated keratinocytes. It is believed that E2 functions as a repressor for P97 transcription after TBP or TFIID binding and its transcriptional repression only occurs in cells harboring integrated, but not episomal HPV-16 DNA. The HPV-16 P670 promoter is a late-promoter and its activity can be induced only in differentiated keratinocytes. Elements in the E6 and E7 coding regions may regulate late promoters and both the late P670 promoter in HPV-16 and P742 in HPV-31 are positioned in the E7 coding region and transcription from the late promoter has to bypass the early pA site to allow expression of the late region. See Zheng & Baker, 2006, Papillomavirus genome structure, expression, and post-transcriptional regulation, Front Biosci 11:2286-2302, incorporated by reference.

The promoters may be used in a vector containing a gene for an antiviral, or targetable, endonuclease.

What is claimed is:

1. A composition for treating a tumor, the composition comprising:
a cancer therapeutic; and
a nuclease in an appropriate diluent, adjuvant or carrier.

2. The composition of claim 1, wherein the nuclease is selected from the group consisting of an endonuclease, an exonuclease, DNase I, a CRISPR-associated nuclease, Cfp1, a transcription-activator-like effector nuclease, a meganuclease, and a zinc-finger nuclease.

3. The composition of claim 1, wherein the cancer therapeutic is selected from the group consisting of actinomycin, all-trans retinoic acid, anthracycline, bleomycin, bortezomib, carboplatin, carfilzomib, capecitabine, cisplatin, chlorambucil, cyclophosphamide, cytarabine, daunorubicin, disulfiram, docetaxel, doxifluridine, doxorubicin, epirubicin, epothilone, epoxomicin, etoposide, fluorouracil, gemcitabine, hydroxyurea, idarubicin, imatinib, interferon alpha, irinotecan, ixazomib, lactacystin, mechlorethamine, mercaptopurine, methotrexate, mitoxantrone, oxaliplatin, paclitaxel, pemetrexed, salinosporamide A, teniposide, topotecan, valrubicin, vinblastine, vincristine, vindesine, and vinorelbine.

4. The composition of claim 1, wherein the nuclease preferentially cuts nucleic acid of a an oncovirus.

5. The composition of claim 4, wherein the nuclease comprises a CRISPR-associated nuclease, and the composition further comprises a guide RNA complementary to a portion of the nucleic acid.

6. The composition of claim 5, wherein the oncovirus is selected from the group consisting of a human papilloma virus (HPV), an Epstein-Barr virus (EBV), Kaposi's sarcoma-associated herpesvirus (KSHV), hepatitis B virus (HBV), hepatitis C virus (HCV), human T-cell lymphotrophic virus type I (HTLV-I), and Merkel cell polyomavirus (MCV).

7. The composition of claim 6, wherein the cancer therapeutic comprises a proteasome inhibitor.

8. The composition of claim 7, wherein the proteasome inhibitor comprises one selected from the group consisting of lactacystin, bortezomib, disulfiram, salinosporamide A, carfilzomib, epoxomicin, and ixazomib.

9. The composition of claim 8, wherein the nuclease is Cas9 and the oncovirus is Epstein-Barr virus.

10. The composition of claim 9, wherein the cancer therapeutic is bortezomib.

11. The composition of claim 4, wherein the cancer therapeutic comprises a monoclonal antibody.

12. The composition of claim 11, wherein the monoclonal antibody is selected from the group consisting of rituximab, bevacizumab, and pembrolizumab.

13. The composition of claim 4, wherein the cancer therapeutic comprises an immune checkpoint inhibitor.

14. The composition of claim 13, wherein the immune checkpoint inhibitors is selected from the group consisting of an anti-PD-1 compound and an anti-VEGF compound.

15. The composition of claim 4, wherein the cancer therapeutic comprises a recombinant cytokine.

16. The composition of claim 15, wherein the recombinant cytokine is selected from the group consisting of Interleukin 2 (IL-2), Interleukin 11 (IL-11), and Interleukin 15 (IL-15).

17. The composition of claim 1, further comprising an antiviral treatment selected from the group consisting of ganciclovir and Gardasil.

18. The composition of claim 1, further comprising an epigenetic modifier.

19. The composition of claim 18, wherein the epigenetic modifier comprises a DNA methyltransferase (DNMT) inhibitor.

20. The composition of claim 18, wherein the epigenetic modifier comprises a histone deacetylase inhibitor.

21. A composition comprising:
a cancer therapeutic and a vector encoding a nuclease, wherein the cancer therapeutic and the nuclease are as described in any of claims 1-20.

22. A method for treating cancer, the method comprising delivering to a tumor:
a cancer therapeutic; and
a nuclease.

23. The method of claim 22, wherein the nuclease is selected from the group consisting of an endonuclease, an exonuclease, DNase I, a CRISPR-associated nuclease, Cfp1, a transcription-activator-like effector nuclease, a meganuclease, and a zinc-finger nuclease.

24. The method of claim 22, wherein the cancer therapeutic is selected from the group consisting of actinomycin, all-trans retinoic acid, anthracycline, bleomycin, bortezomib, carboplatin, carfilzomib, capecitabine, cisplatin, chlorambucil, cyclophosphamide, cytarabine, daunorubicin, disulfiram, docetaxel, doxifluridine, doxorubicin, epirubicin, epothilone, epoxomicin, etoposide, fluorouracil, gemcitabine, hydroxyurea, idarubicin, imatinib, interferon alpha, irinotecan, ixazomib, lactacystin, mechlorethamine, mercaptopurine, methotrexate, mitoxantrone, oxaliplatin, paclitaxel, pemetrexed, salinosporamide A, teniposide, topotecan, valrubicin, vinblastine, vincristine, vindesine, and vinorelbine.

25. The method of claim 22, wherein the nuclease preferentially cuts nucleic acid of an oncovirus.

26. The method of claim 25, wherein the nuclease comprises a CRISPR-associated nuclease, and the method further comprises delivering to the tumor a guide RNA complementary to a portion of the nucleic acid.

27. The method of claim 26, wherein the oncovirus is selected from the group consisting of a human papilloma virus (HPV), an Epstein-Barr virus (EBV), Kaposi's sarcoma-associated herpesvirus (KSHV), hepatitis B virus (HBV), hepatitis C virus (HCV), human T-cell lymphotrophic virus type I (HTLV-I), and Merkel cell polyomavirus (MCV).

28. The method of claim 27, wherein the cancer therapeutic comprises a proteasome inhibitor.

29. The method of claim 28, wherein the proteasome inhibitor comprises one selected from the group consisting of lactacystin, bortezomib, disulfiram, salinosporamide A, carfilzomib, epoxomicin, and ixazomib.

30. The method of claim 26, wherein the cancer therapeutic is bortezomib, the nuclease is Cas9, and the oncovirus is Epstein-Barr virus.

31. The method of claim 26, further comprising an antiviral treatment selected from the group consisting of ganciclovir and Gardasil.

32. The method of claim 26, further comprising an epigenetic modifier.

33. The method of claim 32, wherein the epigenetic modifier comprises a DNA methyltransferase (DNMT) inhibitor.

34. The method of claim 32, wherein the epigenetic modifier comprises a histone deacetylase inhibitor (HDI).
